(19) United States
(12) Patent Application Publication
(10) Pub. No.: US 2004/0110687 A1
(43) Pub. Date: Jun. 10, 2004

(54) Methods and compositions for treating gastrointestinal toxicity induced by cytoablative therapy

(76) Inventor: Roland Buelow, Palo Alto, CA (US)

Correspondence Address: DORSEY & WHITNEY LLP, INTELLECTUAL PROPERTY DEPARTMENT, 4 EMBARCADERO CENTER, SUITE 3400, SAN FRANCISCO, CA, 94111, US

(21) Appl. No.: 10/376,647

(22) Filed: Feb. 26, 2003

Related U.S. Application Data
(60) Provisional application No. 60/360,211, filed on Feb. 26, 2002.

(51) Int. Cl. A61K 38/08

(52) U.S. Cl. 514/15

(57) ABSTRACT

The present invention relates to pharmaceutical preparations and methods for alleviating and reducing the gastrointestinal toxicity and dysfunction induced by cytoablative therapy, wherein immunomodulatory peptides are employed either alone or in combination with additional therapeutic agents. Also provided are improvements to cytoablative cancer therapy, wherein administration of the subject peptides in conjunction with cytoablative therapy enables the administration of increased maximum tolerated doses of the cytoablative agent(s) for improved efficacy and tumor response.

### CROSS-REFERENCE TO RELATED APPLICATIONS

This application claims benefit of U.S. Provisional Patent Application No. 60/360,211 filed Feb. 26, 2002, the entire contents of which is incorporated herein by reference.

### FIELD OF THE INVENTION

The field of this invention relates to novel treatments for individuals suffering from gastrointestinal complications of chemotherapy and radiotherapy, including diarrhea, mucositis, stomatitis and proctitis.

### BACKGROUND OF THE INVENTION

Over the past several decades, potentially curative chemotherapy and radiotherapy techniques have been developed for the treatment of almost all human tumors. Although they have measurably improved survival time and decreased cancer mortality, major toxicities induced by these therapies still represent the rate-limiting step in achieving a cure. Wilmore, Cancer 79:1794-1803 (1997).

Chemotherapeutics and radiation exert their cytoablative effects on rapidly-proliferating cells via several different mechanisms, ultimately leading to cell cycle arrest and/or cellular apoptosis. The cytotoxic actions of these therapies are not tumor specific, however, and normal tissues that typically exhibit rapid cell turnover are the most sensitive to the toxic effect of chemotherapy. In particular, injury to normal cells in the bone marrow and gastrointestinal mucosa often complicates patient treatment. Although significant advances have been made in combating the myelosuppression and neutropenia brought on by bone marrow toxicity, e.g. through the use of recombinant colony-stimulating factors such as Neupogen® and Epogen®, no agents are currently available that can selectively prevent cytoablative gastrointestinal injury. Boushey et al., Cancer Res. 61:687-93 (2001). As a result, gastrointestinal toxicity characterized by severe mucositis and diarrhea often limits both the dose and duration of cytoablative therapy, leading to reduced effectiveness in susceptible patients. Id. The gastrointestinal tract therefore represents the next rate-limiting organ preventing further dose escalation in many patients with cancer. Id.; Wilmore, supra.

The epithelial lining of the intestinal mucosa is maintained by continuous proliferation of cells in crypts, which are ultimately dependent on crypt stem cells. This proliferation is followed by cellular differentiation and migration up the villi, where the mature enterocytes are replaced and shed from the tips. Cytoablative doses of chemotherapy or radiotherapy compromise the absorptive and barrier action of the mucosa by killing the crypt stem cells, thereby impairing normal regeneration. Farrell et al., Cancer Res. 58:933-39 (1998). As the damaged cells slough, the mucosa becomes thin and denuded, accompanied by delayed cellular renewal, mucosal atrophy, inflammation and often ulceration. Balsari et al., Br. J. Cancer 85:1964-67 (2001).

Hence patients undergoing cytoablative therapies frequently develop enteric mucositis and diarrhea, which can be lethal. Cascinu, Curr. Opin. Oncol. 7:325-29 (1995). Moreover, the gastrointestinal effects of these cytoablative therapies can be aggravated and prolonged by the lack of enteral intake that frequently occurs. Anorexia, mucositis, abdominal cramps, explosive diarrhea with food intake and the reliance on intravenous therapy (which suppresses appetite) all compromise the exposure of the gut to enteral nutrients, thus limiting the body's ability to stimulate normal intestinal epithelial proliferation. Wilmore, supra. Moreover, in patients with severe granulocytopenia the ulcerations caused by mucositis can lead to widespread hematogenous bacterial dissemination, since the lesions serve as entry for bacteria into the bloodstream. Balsari et al., supra. Hospitalization is often necessary to maintain body hydration, to control pain, and to prevent or manage infection.

With combination cytoablative therapy in particular, gastrointestinal symptoms may become so severe that the treatment must be modified or discontinued altogether, thus compromising the efficacy of the therapy on the neoplastic disease. In adjunctive therapy, the ability to minimize or attenuate severe gastrointestinal symptoms may greatly influence both quality of life during therapy as well as overall patient survival. This is particularly true with dose escalation therapies used in patients with gastrointestinal cancers or those undergoing bone marrow transplantation associated with high dose combination cytoablative therapy. Wilmore, supra.

Unfortunately, however, there are no universally recognized agents available for prophylaxis or therapy of the gastrointestinal toxicities induced by chemotherapy and/or radiotherapy. Boushey et al., supra. Agents which are commonly employed for treatment of one gastrointestinal complication, e.g., high-dose loperamide for diarrhea, typically have little or no effect on other serious toxicities, e.g. mucositis. Moreover, the therapeutic effects obtained with existing agents directed to specific symptoms such as diarrhea and mucositis are insufficient. What is needed, therefore, is an agent that is capable of alleviating the gastrointestinal toxicities induced by cytoablative therapy.

Relevant Literature

Wilmore, supra, reviews the effects of cytotoxic therapy on the gastrointestinal tract and describes metabolic means of support. Protection against the gastrointestinal toxicity caused by irinotecan has been attempted using thalidomide (Govindarajan et al., Lancet 356:566-7 (2000), interleukin 15 (Cao et al., Cancer Res. 58:3270-74 (1998); a synthetic bacterial lipopeptide (Shinohara et al., Clin. Cancer Res., 5:2148-56 (1999) and glucagon-like peptide (GLP)-2 (Boushey et al., supra). Use of keratinocyte growth factor to protect against gastrointestinal injury caused by combined chemotherapy and radiation regimens is described in Farrell et al., supra, while Orazi et al. disclose the use of interleukin-11. Lab. Invest. 75:33-42 (1996). Protection against adverse gastrointestinal effects of doxorubicin using chitosan is described by Kimura et al., J. Pharm. Pharmacol. 53:1373-78 (2001), while Balsari et al., Br. J. Cancer 85:1964-7 (2001) disclose the use of a doxorubicin-specific monoclonal antibody. Woo et al., infra, describe the use of clarithromycin to attenuate cyclophosphamide-induced mucositis in mice.

Buelow et al., Transplantation 59:649-654 (1995) and references cited therein. Manolios et al., Nature Medicine 3:84-88 (1997) describes oligopeptides derived by rational design which modulate T cell activity. WO 95/13288 by Clayberger et al. which describes peptides capable of modulating T cell activity. References describing methods for designing compounds by computer using structure activity relationships include Grassy et al., J. of Molecular Graphics 13:356-367 (1995); Haiech et al., J. of Molecular Graphics 13:46-48 (1995); Yasri et al., Protein Engineering 11: 959-976 (1996); Ashton et al., Drug Discovery Today 1:71-78 (1996); and Iyer et al., Curr. Pharm. Des. 8:2217-2229 (2002).

### SUMMARY OF THE INVENTION

The present invention relates to pharmaceutical preparations and methods for treating and alleviating gastrointestinal toxicity and dysfunction resulting from intensive cytoablative therapies. In particular, the methods and compositions disclosed herein provide effective prophylaxis and therapy of the gastrointestinal complications induced by chemotherapy and radiotherapy including diarrhea, mucositis (e.g., oral and esophageal), stomatitis and proctitis. The methods and compositions provided herein significantly improve quality of life during ongoing cancer therapies, and enable increased dosages of chemotherapeutic agents and radiotherapy exposures to maximize killing of metastatic cells for improved long-term survival.

In one embodiment, a method for reducing the gastrointestinal toxicity and dysfunction induced by cytoablative therapy is provided, comprising the administration of immunomodulatory peptides either alone or in combination with additional therapeutic agents, e.g., anti-inflammatory agents, anti-diarrheal agents, analgesics and the like. When used in combination with additional therapeutic agents, the immunomodulatory peptides and additional agents may be administered either simultaneously or sequentially. In a particularly preferred embodiment, the immunomodulatory peptides are administered in combination with anti-diarrheal agents such as loperamide.

In a further embodiment, pharmaceutical preparations and kits are provided comprising a novel combination of the immunomodulatory peptides described herein together with at least one additional therapeutic agent including, e.g., anti-inflammatory agents, anti-diarrheal agents, analgesics, and the like.

In another embodiment, the invention provides methods for increasing the maximum tolerated dosage of cytoablative therapies, e.g. chemotherapy and radiotherapy, comprising the administration of immunomodulatory peptides to reduce dose-limiting gastrointestinal toxicities. The dose of immunomodulatory peptide is preferably effective to increase the maximum tolerated dosage (MTD) of cytoablative therapy by at least a quarter (1.25×) or a third (1.33×), more preferably by a half (1.5×), most preferably by 1.5× to 2× or more. In a further embodiment, improved methods of treating cancer are provided employing these increased dosages of chemotherapy and radiotherapy, along with novel combination therapies for alleviation of associated toxicities.

Suitable immunomodulatory peptides for use in the subject compositions and methods are capable of modulating the activity of various immune system cells, particularly T cells, and/or inhibiting the production of inflammatory cytokines. In a preferred embodiment, the subject peptides comprise one or more of the cytomodulating peptides disclosed in co-pending U.S. Patent Applications U.S. Ser. No. 09/028,083 & U.S. Ser. No. 08/838,918 as well as corresponding International Publication WO 98/46633, the disclosures of which are expressly incorporated herein by reference. In a particularly preferred embodiment, the immunomodulating peptide comprises the sequence Arg-nL-nL-nL-Arg-nL-nL-nL-Gly-Tyr, where nL is norleucine and all amino acids are the D-stereoisomer (also referred to herein as bc 1 nL and/or RDP58).

In one aspect, the peptides have amino acid extensions at the N- or C-terminus to provide additional functionality, such as targeting the peptide to the affected tissue, increasing half-life, or for attachment of various compounds. In another aspect, the cytomodulating peptides are oligomers, particularly dimers of the active sequence, or are in the form of cyclic peptides. The peptides may comprise naturally-occurring amino acids or, more preferably, one or more D-stereoisomers.

### BRIEF DESCRIPTION OF THE DRAWINGS

FIG. 1 is a depiction of the conformational space clustering of the bc 1 nL peptide, also referred to herein as RDP58. The conformations drawn are obtained from cluster analysis of bc 1 nL trajectory.

FIG. 2 is a depiction of a projection of peptide trajectories into the principal plan of D2 peptide reference trajectory.

FIG. 3 is a graph showing the dose-dependent effect of RDP58 peptide administration on mortality in a murine tumor model utilizing CPT-11.

FIG. 4 is a graph showing the effect of RDP58 peptide administration in reducing mortality in a murine tumor model utilizing 5-FU.

FIG. 5 is a graph demonstrating the preservation of anti-tumor efficacy when RDP58 peptide is administered in combination with CPT-11 in a murine tumor model.

FIG. 6 is a graph showing the effect of RDP58 peptide administration in reducing mortality in a murine tumor model utilizing CPT-11.

FIG. 7 is a graph illustrating the significant increase in the maximum tolerated dose of CPT-11 enabled by RDP58 peptide administration in a murine tumor model.

FIG. 8 is a graph showing the improved survival of tumor-bearing mice administered an increased dose of CPT-11 in combination with RDP58 peptide.

FIG. 9 is a bar graph illustrating the improved tumor response obtained with the combination of CPT-11 dose escalation and RDP58 peptide treatment.

### DETAILED DESCRIPTION OF THE INVENTION

Methods and compositions are provided for treating major gastrointestinal complications induced by chemotherapy and/or radiotherapy, including diarrhea, mucositis, stomatitis and proctitis. The methods involve the oral administration of members of a new class of synthetic peptides developed by computer-aided rational design from known HLA-derived molecules. See, e.g., U.S. patent application Ser. No. 09/028,083 and International Publication No. WO 98/46633, the disclosures of which are expressly incorporated by reference herein. These new peptides have been shown to have distinct immunomodulatory activities including, e.g., effective inhibition of TNF-α production at the translational level (Iyer et al., J. Biol. Chem. 275:17051-57 (2000)) and upregulation of cellular expression of heme oxygenase-1 (HO-1) (Iyer et al. 1998; Cuturi et al., Mol. Med. 5:820-32 (1999)). The lead compound, identified herein as RDP58, is a ten-amino acid peptide consisting of D-amino acids with the following sequence: Arg-nL-nL-nL-Arg-nL-nL-nL-Gly-Tyr (SEQ ID NO:), where nL indicates D-norleucine.

As demonstrated herein, oral administration of these new peptide compounds can be efficacious in alleviating a variety of gastrointestinal toxicities induced by cytoablative therapy. Thus, the subject peptides will find use in prophylaxis and therapy of gastrointestinal complications such as diarrhea and mucositis resulting from cytoablative therapies, as well as the cramping, discomfort, dehydration, electrolyte imbalance and secondary infections typically attendant to the primary complication. In a further embodiment, use of the subject peptides advantageously enables dose escalation of the cytoablative agent or combination cytoablative therapies to increase tumor cell kill and thereby improve overall patient survival.

The methods of the present invention will find advantageous use with virtually any cytoablative regimen inducing gastrointestinal toxicity, including radiotherapy and chemotherapeutics. Acute mucositis is common after radiotherapy for head and neck cancers, for example, and this mucosal toxicity is recognized as the principal limiting factor to further treatment intensification. See, e.g., Bensadoun et al., Eur. Arch. Otorhinolaryngol. 258:481-7 (2001). The subject peptides will find use in alleviating the mucositis induced with radiotherapy regimens directed to head and neck cancers, as well as for radiotherapy treatments impinging elsewhere upon the gastrointestinal tract such as irradiation in prostate cancer, which can cause in proctitis in the colorectal portion of the gastrointestinal tract, and whole-body irradiation protocols utilized in bone marrow transplantation.

The subject peptides will also find use in alleviating the gastrointestinal toxicities induced by chemotherapeutic agents, either alone or in combination with radiotherapy. For example, the major dose-limiting toxicity associated with the camptothecan analog irinotecan (CPT-11) is delayed or late-onset diarrhea, which can be extremely severe when associated with neutropenia. Rougier et al., J. Clin. Oncol. 15:251-60 (1997). In up to 30% of patients the diarrhea does not respond to conventional agents such as loperamide, and hospital admission, dose modification and/or interruption of chemotherapy is required. Cunningham et al., Lancet 352:1413-18 (1998). As demonstrated herein, oral administration of the subject peptides is effective in alleviating this toxicity.

The subject peptides will also find use in treating mucositis induced by other commonly-used chemotherapeutic agents, including, e.g., methotrexate, 5-fluorouracil, cyclophosphamide, and doxorubicin, as well as taxanes and vinca alkaloids. Severe gastrointestinal mucositis in patients undergoing cytoablative therapy causes significant morbidity and mortality, due to the mucositis itself as well as secondary local and systemic infections predisposed by it. Woo et al., Pharm. Res. 41:527-32 (2000). Despite years of study, the mucositis treatments developed thus far have not had sufficient beneficial effects. Id. As a result, mucositis also remains a primary dose-limiting toxicity associated with many types of chemotherapeutic agents, including doxorubicin which is widely used in treating many types of solid tumors. See Marina et al., Clin. Cancer Res. 8:413-8 (2002). Prophylactic and/or therapeutic treatment of mucosal injury using the subject peptides allows for increased dosages of these agents as well as more effective dosing schedules which may be contra-indicated due to gastrointestinal toxicity

As described herein, administration of the subject peptides alleviates the gastrointestinal toxicities and dysfunction resulting from these intensive cytoablative therapies. Reducing or alleviating gastrointestinal toxicity and dysfunction in the context of cytoablative treatment includes as non-limiting examples, reduction in clinical manifestations such as diarrhea, mucositis, stomatitis, proctitis, rectal bleeding, malabsorption, abdominal pain, weight loss, fever, anemia, fecal occult blood, fecal leukocytes, and histological indications such as crypt abcesses, leukocyte infiltration, cell apoptosis, transmural granulamotous inflammation, superficial mucosal and submucosal inflammation, etc. Furthermore,. included within the definition of symptoms as used herein are changes in levels of biochemical and molecular markers associated with intestinal dysfunction and inflammation arising from cytoablative therapy, including, but not limited to, increase in pro-inflammatory cytokines (e.g., TNF-α, interferon-γ, II-1, IL-6, IL-12, etc.), changes in enzyme markers of leukocyte activation (e.g., myeloperoxidase, COX-2 expression, iNOS expression, etc.), cellular apoptosis (e.g., DNA fragmentation, caspase activation, etc.), and others known in the art. Although one marker may be used as an indication of reduction in gastrointestinal dysfunction, preferably more than one is used, and more preferably a combination of markers is used, including combinations of clinical manifestations, histological indications, and molecular/biochemical markers.

For use in alleviating and/or reducing the gastrointestinal toxicities and dysfunction of patients undergoing cytoablative treatment, the immunomodulatory peptides may be used alone or in combination with other therapeutic agents, such as, e.g., oxygen radical scavenging agents such as superoxide dismutase or anti-inflammatory agents such as corticosteroids, hydrocortisone, prednisone and the like; anti-diarrheal agents such as loperamide and the like, antibacterial agents such as penicillin, cephalosporins, bacitracin and the like; antiparasitic agents such as quinacrine, chloroquine and the like; antifungal agents such as nystatin, gentamicin, and the like; antiviral agents such as acyclovir, gancyclovir, ribavirin, interferons and the like; analgesic agents such as salicylic acid, acetaminophen, ibuprofen, flurbiprofen, morphine and the like; local anesthetics such as lidocaine, bupivacaine, benzocaine and the like; growth factors such as colony stimulating factor, granulocyte-macrophage colony stimulating factor, and the like; antihistamines such as diphenhydramine, chlorphencramine and the like; anti-nausea medications, nutritional additives such as leukovorin, and other like substances.

Thus, a plurality of therapeutic agents may be used in the present invention. These multi-drug combinations, include, but are not limited to, combinations of the subject peptides with anti-diarrheal agents, anti-inflammatory agents and/or analgesics. For example, one embodiment may comprise a combination containing the immunomodulatory peptides disclosed herein, particularly the D-stereo isomer of sequence Arg-nL-nL-nL-Arg-nL-nL-nL-Gly-Tyr; loperamide, and an analgesic. Other combinations may be made by those skilled in the art (e.g., different anti-inflammatory agents and analgesic combinations). In this context, the peptides used are either a single peptide sequence, or an admixture of different peptide sequences of the present invention, or an admixture that includes natural analogs of the peptides of the present invention (e.g., B2702.75-84).

The methods and compositions described herein may find advantageous use in reducing or alleviating the gastrointestinal toxicity and/or dysfunction caused by any form of cytoablative therapy, and in particular, those therapies targeting rapidly-dividing cells including both tumor cells and epithelial cells. Non-limiting examples of potential antineoplastic agents that may benefit from the subject invention include platinum compounds (e.g., spiroplatin, cisplatin, and carboplatin), camptothecan and related analogs such as irinotecan (CPT-11), taxanes such as taxol, mitotic inhibitors such as etoposide and the vinca alkaloids including, e.g., vincristine, vinblastine and vinorelbine, methotrexate, fluorouracil (5-FU), adriamycin, mitomycin, ansamitocin, bleomycin, cytosine arabinoside, arabinosyl adenine, mercaptopolylysine, busulfan, chlorambucil, melphalan (e.g., PAM, L-PAM or phenylalanine mustard), mercaptopurine, mitotane, procarbazine hydrochloride dactinomycin (actinomycin D), daunorubicin hydrochloride, doxorubicin hydrochloride, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), interferon .alpha.-2a, interferon .alpha.-2b, teniposide (VM-26), vinblastine sulfate (VLB), vincristine sulfate, bleomycin sulfate, arabinosyl, hydroxyurea, procarbazine, and dacarbazine; as well as radiopharmaceuticals such as radioactive iodine and phosphorus products and the like.

Immunomodulatory Peptides

Immunomodulatory peptides suitable for use in the compositions and methods of the present invention are capable of inhibiting the cellular production of inflammatory cytokines including, e.g., tumor necrosis factor-α (TNF-α), interferon-γ (INF-γ), interleukin (IL)-1, IL-8, IL-12 as well as other cytokines, chemokines, hematopoietic growth factors, and the like. Preferred immunomodulatory peptides include or comprise one or more of the cytomodulating oligopeptides described in co-pending U.S. patent applications Ser. No. 08/838,916 and U.S. Ser. No. 09/028,083, the disclosures of which are incorporated by reference herein. Particularly preferred for use in the instant methods and compositions is an immunomodulatory peptide comprising the sequence Arg-nL-nL-nL-Arg-nL-nL-nL-Gly-Tyr, where nL is norleucine and all amino acids are the D-stereoisomer.

In addition, previously-known active compounds which may also find use in the subject invention include HLA-B α₁-domain, particularly the amino acids from 75 to 84 and variations of this sequence where not more than 2 amino acids are replaced and in which amino acids do not include R and Y (see, e.g., WO 95/13288 and Buelow et al., supra). Also known are sequences based on the human TCR-α transmembrane region consisting of that sequence and sequences having not more than 2 mutations from that sequence (see Australian Application Nos. PN 0589 and PN 0590, filed Jan. 16, 1995). These sequences include 2 basic amino acids, where the 2 basic amino acids are separated by 4 aliphatic hydrophobic amino acids, although the application indicates that from 3 to 5 hydrophobic amino acids may be present. By mutation is intended each substitution of one amino acid for another or an insertion or deletion, each being counted as one mutation. In certain embodiments, the immunostimulatory peptides preferred for use herein may exclude such previously-known active compounds.

Generally, the phrase “immunomodulatory peptides” or “immunomodulating peptides” as used herein is meant to encompass all of the foregoing peptide compounds, as well as analogs, derivatives, fusion proteins and the like. In the preferred embodiment, the core sequence of the immunomodulatory peptide desirably comprises two basic amino acids separated by from three to four hydrophobic amino acids, particularly three hydrophobic amino acids, and particularly where the N-terminus is a basic amino acid. More desirably, the C-terminal amino acid is an aromatic amino acid, particularly tyrosine. Of particular interest is where at least one of the oligopeptide core terminal amino acids is an oligopeptide terminal amino acid, which may be in the monomeric or oligomeric form of the compound.

More particularly, the preferred immunomodulatory peptides for use in the compositions and methods of the present invention comprise oligopeptides having the sequence B-X-X-X-B-X-X-X-J-Tyr, where B is a basic amino acid, preferably Lys or Arg, particularly Arg on at least one position, preferably at both positions; J is Gly, B or an aliphatic hydrophobic amino acid of from 5 to 6 carbon atoms, particularly Gly or B; and X is an aliphatic or aromatic amino acid. In one embodiment, at least three X amino acid residues are the same non-polar aliphatic amino acid, preferably at least four are the same non-polar aliphatic amino acid, more preferably at least five are the same non-polar aliphatic amino acid, and most preferably, all are the same non-polar aliphatic amino acid. In a preferred embodiment, the non-polar aliphatic amino acids are of from 5 to 6 carbon atoms, particularly 6 carbon atoms, particularly the non-polar aliphatic amino acids Val, Ile, Leu, and nL. Thus, in some embodiments, X is any amino acid other than a charged aliphatic amino acid, and preferably any amino acid other than a polar aliphatic amino acid.

Of the six amino acids indicated by X in the B-X-X-X-B-X-X-X-J-Tyr peptide sequence, preferably at least 3 are aliphatic amino acids of from 5 to 6 carbon atoms, more preferably at least 4 are aliphatic amino acids of from 5 to 6 carbon atoms, most preferably at least 5 are aliphatic amino acids of 5-6 carbon atoms, more particularly 6 carbon atoms. In a preferred embodiment, the aliphatic amino acids are non-polar aliphatic amino acids of from 5 to 6 carbon atoms, particularly Val, Ile, Leu, and nL. The other amino acids may be other uncharged aliphatic amino acids, particularly non-polar aliphatic amino acids or aromatic amino acids.

Compositions of particular interest will have the following formula:

Arg-U-X-X-Arg-X-X-X-J-Tyr

wherein all of the symbols have been defined previously except U, which comprises an uncharged aliphatic amino acid or aromatic amino acid, particularly a non-polar aliphatic amino acid or aromatic amino acid.

The amino acids may be naturally occurring amino acids or D- isomers thereof. The peptides may have one or more D-stereoisomer amino acids, up to all of the amino acids. Additionally, the immunomodulatory peptides may comprise oligomers of the subject peptides, particularly dimers thereof, or comprise a cyclic peptide, that is a ring structure, as further described below.

For the purposes of this invention, the amino acids (for the most part natural amino acids or their D-stereoisomers) will be broken down into the following categories:

1. Aliphatic

(a) non-polar aliphatic:

Gly, Ala, Val, nL, Ile, Leu

(b) polar aliphatic:

(1) uncharged:

Cys, Met, Ser, Thr, Asn, Gln

(2) charged:

Asp, Glu, Lys, Arg

2. Aromatic:

Phe, His, Trp, Tyr

wherein Pro may be included in the non-polar aliphatic amino acids, but will normally not be included. “nL” represents norleucine, where the non-polar aliphatic amino acids may be substituted with other isomers.

Either or both the N- and C-terminus of the peptide may be extended by not more than a total of about 100, usually not more than a total of about 30, more usually not more than about 20 amino acids, often not more than about 9 amino acids, where the amino acids will have fewer than 25%, more usually fewer than 20% polar amino acids, more particularly, fewer than 20% which are charged amino acids. Thus, extensions of the above sequences in either direction are mainly done with lipophilic, uncharged amino acids, particularly non-polar aliphatic amino acids and aromatic amino acids. The peptides may comprise L-amino acids, D-amino acids, or mixtures of D and L amino acids. Exceptions to the number of amino acid extensions are contemplated when the oligopeptides are expressed as fusion or chimeric proteins, as described below.

The peptides may be in the form of oligomers, particularly dimers of the peptides, which may be head to head, tail to tail, or head to tail, there being not more than about 6 repeats of the peptide. The oligomer may contain one or more D-stereoisomer amino acids, up to all of the amino acids. The oligomers may or may not include linker sequences between the peptides. When linker sequences are used, suitable linkers include those comprising uncharged amino acids and (Gly)n, where n is 1-7, Gly-Ser (e.g., (GS)ₙ, (GSGGS)ₙ and (GGGS)ₙ, where n is at least 1), Gly-Ala, Ala-Ser, or other flexible linkers, as known in the art. Linkers of Gly or Gly-Ser may be used since these amino acids are relatively unstructured, which allows interaction of individual peptides with cellular target molecules and limits structural perturbations between peptides of the oligomer.

Immunomodulatory peptides may be in a structurally constrained form such as cyclic peptides of from about 9-50, usually 12 to 36 amino acids, where amino acids other than the specified amino acids may be present as a bridge. Thus, for example, addition of terminal cysteines allows formation of disulfide bridges to form a ring peptide. In some instances, one may use other than amino acids to cyclize the peptide. Bifunctional crosslinking agents are useful in linking two or more amino acids of the peptide. Other methods for ring formation are described in Chen et al., Proc. Natl. Acad. Sci. USA 89:5872-5876 (1992); Wu et al., Protein Engineering 6:471-478 (1993); Anwer, et al., Int. J. Pep. Protein Res. 36:392-399 (1990); and Rivera-Baeza, et al. Neuropeptides 30: 327-333 (1996); all references incorporated by reference. Alternatively, structurally constrained peptides are made by addition of dimerization sequences to the N- and C-terminal ends of the peptide, where interaction between dimerization sequences lead to formation of a cyclic type structure (see WO/0166565, incorporated by reference). In other instances, the subject peptides are expressed as fusions to other proteins, which provide a scaffold for constrained display on a surface exposed structure, such as a loop of a coiled-coil or β-turn structure.

One or both, usually one terminus of the immunomodulatory peptide, may be substituted with a lipophilic group, usually aliphatic or aralkyl, of from 8 to 36, usually 8 to 24 carbon atoms and fewer than two heteroatoms in the aliphatic chain, the heteroatoms usually being oxygen, nitrogen and sulfur. As further described below, the chain may be saturated or unsaturated, desirably having not more than 3 sites, usually not more than 2 sites of aliphatic unsaturation. Conveniently, commercially available aliphatic fatty acids, alcohols and amines may be used, such as caprylic acid, capric acid, lauric acid, myristic acid and myristyl alcohol, palmitic acid, palmitoleic acid, stearic acid and stearyl amine, oleic acid, linoleic acid, docosahexaenoic acid, etc. (see U.S. Pat. No. 6,225,444, hereby incorporated by reference). Preferred are unbranched, naturally occurring fatty acids between 14-22 carbon atoms in length. Other lipohilic molecules include glyceryl lipids and sterols, such as cholesterol. The lipophilic groups may be reacted with the appropriate functional group on the oligopeptide in accordance with conventional methods, frequently during the synthesis on a support, depending on the site of attachment of the oligopeptide to the support. Lipid attachment is useful where oligopeptides may be introduced into the lumen of the liposome, along with other therapeutic agents (e.g., immunosuppressive agents) for administering the peptides and agents into a host. Increasing lipophilicity is also known to increase transport of compounds across endothelial cells and therefore useful in promoting uptake of such compounds from the intestine or blood stream into surrounding tissues.

The terminal amino group or carboxyl group of the immunomodulatory peptide may be modified by alkylation, amidation, or acylation to provide esters, amides or substituted amino groups, where the alkyl or acyl group may be of from about 1 to 30, usually 1 to 24, preferably either 1 to 3 or 8 to 24, particularly 12 to 18 carbon atoms. The peptide or derivatives thereof may also be modified by acetylation or methylation to alter the chemical properties, for example lipophilicity. Other modifications include deamination of glutamyl and asparaginyl residues to the corresponding glutamyl and aspartyl residues, respectively; hydroxylation of proline and lysine; phosphorylation of hydroxyl groups of serine or threonine; and methylation of amino groups of lysine, arginine, and histidine side chains (see T. E. Creighton, Proteins: Structure and Molecular Properties, W.H. Freeman & Co. San Francisco, Calif., 1983).

Depending upon their intended use, particularly for administration to mammalian hosts, the subject peptides may be modified or attached to other compounds for the purposes of incorporation into carrier molecules, changing peptide bioavailability, extend or shorten half-life, control distribution to various tissues or the blood stream, diminish or enhance binding to blood components, and the like. The subject peptides may be bound to these other components by linkers which are cleavable or non-cleavable in the physiological environment such as blood, cerebrospinal fluid, digestive fluids, etc. The peptides may be joined at any point of the peptide where a functional group is present, such as hydroxyl, thiol, carboxyl, amino, or the like. Desirably, modification will be at either the N-terminus or the C-terminus. For these purposes, the subject peptides may be modified by covalently attaching polymers, such as polyethylene glycol, polypropylene glycol, carboxymethyl cellulose, dextran, polyvinyl alcohol, polyvinylpyrrolidine, polyproline, poly(divinyl-ether-co-maleic anhydride), poly(styrene-c-maleic anhydride), etc. Water soluble polymers, such a polyethylene glycol and polyvinylpyrrolidine are known to decrease clearance of attached compounds from the blood stream as compared to unmodified compounds. The modifications can also increase solubility in aqueous media and reduce aggregation of the peptides.

In another aspect, the peptide is preferably conjugated to small molecules for detection and isolation of the peptides, and to target or transport the immunomodulatory peptide into specific cells, tissues, and organs. Small molecule conjugates include haptens, which are substances that do not initiate an immune response when introduced by themselves into an animal. Generally, haptens are small molecules of molecular weight less than about 2 kD, and more preferably less that about 1 kD. Haptens include small organic molecules (e.g., p-nitrophenol, digoxin, heroin, cocaine, morphine, mescaline, lysergic acid, tetrahydrocannabinol, cannabinol, steroids, pentamidine, biotin, etc.). Binding to the hapten, for example for purposes of detection or purification, are done with hapten specific antibodies or specific binding partners, such as avidin which binds biotin.

Small molecules that target the conjugate to specific cells or tissues may also be used. It is known that presence of a biotin-avidin complex increases uptake of such modified peptides across endothelial cells. Linkage of peptides to carbohydrate moieties, for example to a β-glycoside through a serine residue on the peptide to form a β-O linked glycoside, enhances transport of the glycoside derivative via glucose transporters (Polt, R. et al. Proc. Natl. Acad. Sci. USA 91: 7144-7118 (1994); Oh et al. Drug Transport and targeting, In Membrane Transporters as Drug Targets, Amidon, G. L. and Sadee, W. eds., pg 59-88, Plenum Press, New York, 1999). Both of these types of modifications are encompassed within the scope of the present invention.

The immunomodulatory peptides may have attached various label moieties such as radioactive labels and fluorescent labels for detection and tracing. Fluorescent labels include, but are not limited to, fluorescein, eosin, Alexa Fluor, Oregon Green, rhodamine Green, tetramethylrhodamine, rhodamine Red, Texas Red, coumarin and NBD fluorophores, the QSY 7, dabcyl and dabsyl chromophores, BIODIPY, Cy⁵, etc.

In one aspect, the peptides are joined to a wide variety of other peptides or proteins for a variety of purposes. The peptides may be linked to peptides or proteins to provide convenient functionalities for bonding, such as amino groups for amide or substituted amine formation, e.g., reductive amination; thiol groups for thioether or disulfide formation; carboxyl groups for amide formation; and the like. Of particular interest are peptides of at least 2, more usually 3, and not more than about 60 lysine groups, particularly polylysines of from about 4 to 20, usually 6 to 18 lysine units, referred to as multiple antigenic peptide system (MAPS), where the subject peptides are bonded to the lysine amino groups, generally at least about 20%, more usually at least about 50%, of available amino groups, to provide a multipeptide product (Butz, S. et al. Pept. Res. 7: 20-23 (1994)). In this way, molecules having a plurality of the subject peptides are obtained where the orientation of the subject peptides is in the same direction; in effect one has a linking group to provide for tail to tail di -or oligomerization.

In another aspect, other naturally occurring or synthetic peptides and proteins may be used to provide a carrier immunogen for generating antibodies to the subject peptides, where the antibodies serve as reagents for detecting the immunomodulatory peptides or for identifying other peptides having a comparable conformation. Suitable carriers for generating antibodies include, among others, hemocyanins (e.g., Keyhole Limpet hemocyanin—KLH); albumins (e.g., bovine serum albumin, ovalbumin, human serum albumin, etc.); immunoglobulins; thyroglobulins (e.g., bovine thyroglobulin); toxins (e.g., diptheria toxoid, tetanus toxoid); and polypeptides such as polylysine, as described above, or polyalanine-lysine. Although proteins are preferred carriers, other carriers, preferably high molecular weight compounds, may be used, including carbohydrates, polysaccharides, lipopolysaccharides, nucleic acids, and the like of sufficient size and immunogenicity. In addition, the resulting antibodies may be used to prepare anti-idiotypic antibodies which may compete with the subject peptides for binding to a target site. These anti-idiotypic antibodies are useful for identifying proteins to which the subject peptides bind.

In another aspect, the peptides are conjugated to other peptides or proteins for targeting the immunomodulatory peptide to cells and tissues, or adding additional functionalities to the peptides. For targeting, the protein or peptide used for conjugation will be selected based on the cell or tissue being targeted for therapy (Lee, R. et al. Arthritis. Rheum. 46: 2109-2120 (2002); Pasqualini, R. Q. J. Nucl. Med. 43: 159-62 (1999); Pasgualini, R. Nature 380: 364-366 (1996); hereby incorporated by reference). The proteins may also compromise poly-amino acids including, but not limited to, polyarginine; and polylysine, polyaspartic acid, etc., which may be incorporated into other polymers, such as polyethylene glycol, for preparation of vesicles or particles containing the conjugated peptides.

In another aspect, the subject peptides may be expressed in conjunction with other peptides or proteins, so as to be a portion of the polypeptide chain, either internal, or at the N- or C-terminus to form chimeric proteins or fusion proteins. By “fusion polypeptide” or “fusion protein” or “chimeric protein” herein is meant a protein composed of a plurality of protein components that, while typically joined in the native state, are joined by the respective amino and carboxy termini through a peptide linkage to form a continuous polypeptide. Plurality in this context means at least two, and preferred embodiments generally utilize three to twelve components, although more may be used. It will be appreciated that the protein components can be joined directly or joined through a peptide linker/spacer as outlined below.

Fusion polypeptides may be made to a variety of peptides or proteins to display the subject peptides in a conformationally restricted form, for targeting to cells and tissues, for targeting to intracellular compartments, tracking the fusion protein in a cell or an organism, and screening for other molecules that bind the peptides. Proteins useful for generating fusion proteins include various reporter proteins, structural proteins, cell surface receptors, receptor ligands, toxins, and enzymes. Exemplary proteins include fluorescent proteins (e.g., Aequoria victoria GFP, Renilla reniformis GFP, Renilla muelleri GFP, luciferases, etc., and variants thereof); β-galactosidase; alkaline phosphatase; E. coli. maltose binding protein; coat proteins of filamentous bacteriophage (e.g., minor coat protein, pill, or the major coat protein, pVIII, for purposes of phage display); T cell receptor; charybdotoxin; and the like.

Fusion proteins also encompass fusions with fragments of proteins or other peptides, either alone or as part of a larger protein sequence. Thus, the fusion polypeptides may comprise fusion partners. By “fusion partners” herein is meant a sequence that is associated with the peptide that confers all members of the proteins in that class a common function or ability. Fusion partners can be heterologous (i.e., not native to the host cell) or synthetic (i.e., not native to any cell). The fusion partners include, but are not limited to, a) presentation structures, which provide the subject peptides in a conformationally restricted or stable form; b) targeting sequences, which allow localization of the peptide to a subcellular or extracellular compartment; c) stability sequences, which affects stability or protection from degradation to the peptide or the nucleic acid encoding it; d) linker sequences, which conformationally decouples the oligopeptide from the fusion partner; and e) any combination of the above.

In one aspect, the fusion partner is a presentation structure. By “presentation structure” as used herein is meant a sequence that when fused to the subject peptides presents the peptides in a conformationally restricted form. Preferred presentation structures enhance binding interactions with other binding partners by presenting a peptide on a solvent exposed exterior surface, such as a loop. Generally, such presentation structures comprise a first portion joined to the N-terminus of the immunomodulatory peptide and a second portion joined to the C-terminal end of the subject peptide. That is, the peptide of the present invention is inserted into the presentation structures. Preferably, the presentation structures are selected or designed to have minimal biological activity when expressed in the target cells.

Preferably, the presentation structures maximize accessibility to the peptides by displaying or presenting the peptide or an exterior loop. Suitable presentation structures include, but are not limited to, coiled coil stem structures, minibody structures, loops on β-turns, dimerization sequences, cysteine linked structures, transglutaminase linked structures, cyclic peptides, helical barrels, leucine zipper motifs, etc.

In one embodiment, the presentation structure is a coiled-coil structure, which allows presentation of the subject peptide on an exterior loop (see Myszka et al. Biochemistry 33: 2362-2373 (1994)), such as a coiled-coil leucine zipper domain (see Martin et al. EMBO J. 13: 5303-5309 (1994)). The presentation structure may also comprise minibody structures, which is essentially comprised of a minimal antibody complementarity region. The minibody structure generally provides two peptide regions that are presented along a single face of the tertiary structure in the folded protein (see Bianchi et al. J. Mol. Biol. 236: 649-659 (1994); Tramontano et al. J. Mol. Recognit. 7: 9-24 (1994)).

In another aspect, the presentation structure comprises two dimerization sequences. The dimerization sequences, which can be same or different, associate non-covalently with sufficient affinity under physiological conditions to structurally constrain the displayed peptide; that is, if a dimerization sequence is used at each terminus of the subject oligopeptide, the resulting structure can display the subject peptide in a structurally limited form. A variety of sequences are suitable as dimerization sequences (see for example, WO 99/51625; incorporated by reference). Any number of protein-protein interaction sequences known in the art are useful.

In a further aspect, the presentation sequence confers the ability to bind metal ions to generate a conformationally restricted secondary structure. Thus, for example, C2H2 zinc finger sequences are used. C2H2 sequences have two cysteines and two histidines placed such that a zinc ion is chelated. Zinc finger domains are known to occur independently in multiple zinc-finger peptides to form structurally independent, flexibly linked domains (see Nakaseko, Y. et al. J. Mol. Biol. 228: 619-636 (1992)). A general consensus sequence is (5 amino acids)-C-(2 to 3 amino acids)-C-(4 to 12 amino acids)-H-(3 amino acids)-H-(5 amino acids). A preferred example would be -FQCEEC-random peptide of 3 to 20 amino acids-HIRSHTG. Similarly, CCHC boxes having a consensus sequence -C-(2 amino acids)-C-(4 to 20 random peptide)-H-(4 amino acids)-C- can be used, (see Bavoso, A. et al. Biochem. Biophys. Res. Commun. 242: 385-389 (1998)). Other examples include (1) -VKCFNC-4 to 20 random amino acids-HTARNCR-, based on the nucleocapsid protein P2; (2) a sequence modified from that of the naturally occurring zinc-binding peptide of the Lasp-1 LIM domain (Hammarstrom, A. et al. Biochemistry 35: 12723-32 (1996)); and (3) -MNPNCARCG-4 to 20 random amino acids-HKACF-, based on the NMR structural ensemble 1ZFP (Hammarstrom et al., supra).

In yet another aspect, the presentation structure is a sequence that comprises two or more cysteine residues, such that a disulfide bond may be formed, resulting in a conformationally constrained structure. That is, use of cysteine containing peptide sequences at each terminus of the subject immunomodulatory peptides results in cyclic peptide structures, as described above. A cyclic structure reduces susceptibility of the presented peptide to proteolysis and increases accessibility to its target molecules. As will be appreciated by those skilled in the art, this particular embodiment is particularly suited when secretory targeting sequences are used to direct the peptide to the extracellular space.

In another embodiment, the fusion partner is a targeting sequence. Targeting sequences comprise binding sequences capable of causing binding of the expressed product to a predeterimed molecule or class of molecules while retaining bioactivity of the expression product; sequences signaling selective degradation of the fusion protein or binding partners; and sequences capable of constitutively localizing peptides to a predetermined cellular locale. Typical cellular locations include subcellular locations (e.g, Golgi, endoplasmic recticulum, nucleus, nucleoli, nuclear membrane, mitochondria, secretory vesicles, lysosomes) and extracellular locations by use of secretory signals.

Various targeting sequences are known in the art. Targeting to nucleus is achieved by use of nuclear localization signals (NLS). NLSs are generally short, positively charged domains that directs the proteins in which the NLSs is present to the cells nucleus. Typical NLSs sequences include the single basic NLSs of SV40 large T antigen (Kalderon et al. Cell 39: 499-509 (1984)); human retinoic acid receptor-β nuclear localization signal (NF-kB p50 and p65 (Ghosh et al. Cell 62: 1019-1029 (1990)); Nolan et al. Cell 64: 961-999 (1991)); and the double basic NLSs' as exemplified by nucleoplasmin (Dingwall et al. J. Cell Biol. 107: 641-649 (1988)).

In another aspect the targeting sequences are membrane anchoring sequences. Peptides are directed to the membrane via signal sequences and stably incorporated in the membrane through a hydrophobic transmembrane domain (designated as TM). The TM segment is positioned appropriately on the expressed fusion protein to display the subject peptide either intracellularly or extracellularly, as is known in the art. Membrane anchoring sequences and signal sequences include, but are not limited to, those derived from (a) class I integral membrane proteins such as IL-2 receptor β-chain; Hatekeyama et al. Science 244: 551-556 (1989)) and inuslin receptor β-chain (Hetekayama et al, supra); (b) class II integral membrane proteins such as neutral endopeptidase (Malfroy et al Biochem. Biophys. Res. Commun. 144: 59-66 (1987)); and (c) type III proteins such as human cytochrome P450 NF25 (Hetekayama et al, supra); and those from CD8, ICAM-2, IL-8R, and LFA-1.

Membrane anchoring sequences also include the GPI anchor, which results in covalent bond formation between the GPI anchor sequence and the lipid bilayer via a glycosyl-phosphatidylinositol. GPI anchor sequences are found in various proteins, including Thy-1 and DAF (see Homans et al. Nature 333: 269-272 (1988)). Similarly, acylation sequences allow for attachment of lipid moieties, e.g., isoprenylation (i.e., farnesyl and geranyl-geranyl; see Farnsworth et al. Proc. Natl. Acad. Sci. USA 91: 11963-11967 (1994) and Aronheim et al. Cell 78: 949-61 (1994)), myristoylation (Stickney, J. T. Methods Enzymol. 332: 64-77 (2001)), or palmitoylation. In one aspect, the subject peptide will be bound to a lipid group at a terminus, so as to be able to be bound to a lipid membrane, such as a liposome.

Other intracellular targeting sequences are lysozomal targeting sequences (e.g., sequences in LAMP-1 and LAMP-2; Uthayakumar et al. Cell Mol. Biol. Res. 41: 405-420 (1995) and Konecki et al. Biochem. Biophys. Res. Comm. 205: 1-5 (1994)); mitochondrial localization sequences (e.g., mitochondrial matrix sequences, mitochondrial inner membrane sequences, mitochondrial intermembrance sequences, or mitochondrial outer membrane sequences; see Shatz, G. Eur. J. Biochem. 165: 1-6 (1987)); endoplasmic recticulum localization sequences (e.g., calreticulin, Pelham, H. R. Royal Soc. London Transactions B: 1-10 (1992); adenovirus E3/19K protein, Jackson et al. EMBO J. 9: 3153-3162 (1990)); and peroxisome localization sequences (e.g., luciferase peroxisome matrix sequence, Keller et al. Proc. Natl. Acad. Sci. USA 4: 3264-3268 (1987)).

In another aspect, the targeting sequences is a secretory signal sequence which effects secretion of the peptide. A large number of secretory sequences are known to direct secretion of a peptide into the extracellular space when placed at the amino end relative to the peptide of interest, particularly for secretion of a peptide by cells, including transplanted cells. Suitable secretory signals included those found in IL-2 (Villinger et al. J. Immuno. 155: 3946-3954 (1995)), growth hormone (Roskam et al. Nucleic Acids Res. 7: 305-320 (1979)), preproinsulin, and influenza HA protein.

The fusion partner may further comprise a stability sequence, which confers stability to the fusion protein or the nucleic acid encoding it. Thus, for example, incorporation of glycines after the initiating methionine (e.g., MG or MGG) can stabilize or protect the fused peptide from degradation via ubiquitination as per the N-End rule of Varshavsky, thus conferring increased half-life in a cell.

Additional amino acids may be added for tagging the peptide for purposes of detection or purification. These sequences may comprise epitopes recognized by antibodies (e.g., flag tags) or sequences that bind ligands, such a metals ions. Various tag sequences and ligand binding sequences are well known in the art. These include, but is not limited to, poly-histidine (e.g., 6×His tags, which are recognized by antibodies but also bind divalent metal ions); poly-histidine-glycine (poly-his-gly) tags; flu HA tag polypeptide; c-myc tag; Flag peptide (Hopp et al. BioTechnology 6: 1204-1210 (1988)); KT3 epitope peptide; tubulin epitope peptide (Skinner et al. J. Biol. Chem. 266: 15163-12166 (1991)); and T7 gene 10 protein peptide tag (Lutz-Freyermuth et al. Proc. Natl. Acad. Sci. USA 87: 6363-6397 (1990)).

Fusion partners includes linker or tethering sequences for linking the peptides and for presenting the peptides in an unhindered structure. As discussed above, useful linkers include glycine polymers (G)n where n is 1 to about 7, glycine-serine polymers (e.g., (GS)n, (GSGGS)n and (GGGS)n, where n is at least 1), glycine-alanine polymers, alanine-serine polymers, and other flexible linkers known in the art. Preferably, the linkers are glycine or glycine-serine polymers since these amino acids are relatively unstructured, hydrophilic, and are effective for joining segments of proteins and peptides.

In the present invention, combinations of fusion partners may be used. Any number of combinations of presentation structures, targeting sequences, rescue sequences, tag sequences and stability sequences may be used with or without linker sequences.

The immunomodulatory peptides utilized in the methods and compositions of the present invention may be prepared in a number of ways. Chemical synthesis of peptides are well known in the art. Solid phase synthesis is commonly used and various commercial synthetic apparatuses are available, for example automated synthesizers by Applied Biosystems Inc., Foster City, Calif.; Beckman; etc. Solution phase synthetic methods may also be used, although it is less convenient. By using these standard techniques, naturally occurring amino acids may be substituted with unnatural amino acids, particularly D-stereoisomers, and also with amino acids with side chains having different lengths or functionalities. Functional groups for conjugating to small molecules, label moieties, peptides, or proteins, or for purposes of forming cyclized peptides may be introduced into the molecule during chemical synthesis. In addition, small molecules and label moieties may be attached during the synthetic process. Preferably, introduction of the functional groups and conjugation to other molecules minimally affects the structure and function of the subject peptide.

The N- and C-terminus may be derivatized using conventional chemical synthetic methods. The immunomodulatory peptides of the invention may contain an acyl group, such as an acetyl group. Methods for acylating, and specifically for acetylating the free amino group at the N-terminus are well known in the art. For the C-terminus, the carboxyl group may be modified by esterification with alcohols or amidated to form —CONH₂, CONHR, or CONR, wherein each R is a hybroxycarbyl (1-6 carbons). Methods of esterification and amidation are done using well known techniques.

The subject immunomodulatory peptides utilized herein may also be present in the form of a salt, generally in a salt form which is pharmaceutically acceptable. These include inorganic salts of sodium, potassium, lithium, ammonium, calcium, magnesium, iron, zinc, copper, manganese, and the like. Various organic salts of the peptide may also be made with, including, but not limited to, acetic acid, propionic acid, pyruvic acid, maleic acid, succinic acid, tartaric acid, citric acid, benozic acid, cinnamic acid, salicylic acid, etc.

Synthesis of the immunomodulatory peptides and derivatives thereof may also be carried out by using recombinant techniques. For recombinant production, one may prepare a nucleic acid sequence which encodes a single oligopeptide or preferably a plurality of the subject peptides in tandem with an intervening amino acid or sequence, which allows for cleavage to the single peptide or head to tail dimers. Where methionine or tryptophane is absent, an intervening methionine or tryptophane may be incorporated, which allows for single amino acid cleavage using CNBr or BNPS-Skatole (2-(2-nitrophenylsulfenyl)-3-methyl-3-bromoindolenine), respectively. Alternatively, cleavage is accomplished by use of sequences that are recognized by particular proteases for enzymatic cleavage or sequences that act as self-cleaving sites (e.g., 2A sequences of apthoviruses and cardioviruses; Donnelly, M. L. J. Gen. Virol. 78:13-21. (1997); Donnelly, M. L. J. Gen. Virol. 82:1027-41 (2001), hereby incorporated by reference). The subject peptide may also be made as part of a larger peptide, which can be isolated and the oligopeptide obtained by proteolytic cleavage or chemical cleavage. The particular sequence and the manner of preparation will be determined by convenience, economics, purity required, and the like. To prepare these compositions, a gene encoding a particular peptide, protein, or fusion protein is joined to a DNA sequence encoding the immunomodulatory peptides of the present invention to form a fusion nucleic acid, which is introduced into an expression vector. Expression of the fusion nucleic acid is under the control of a suitable promoter and other control sequences, as defined below, for expression in a particular host cell or organism (see, Sambrook et al., Molecular Biology: A Laboratory Manual, 3rd Ed., Cold Spring Harbor Laboratories, Cold Spring Harbor, N.Y., 2001; Ausubel et al. Current Protocols in Molecular Biology, John Wiley & Sons, New York, N.Y., 1988, updates up to 2002; incorporated by reference).

When the synthesis or delivery of the immunomodulatory peptides are via nucleic acids encoding the subject peptides, the nucleic acids are cloned into expression vectors and introduced into cells or a host. The expression vectors are either self-replicating extrachromosomal vectors or vectors that integrate into the host chromosome, for example vectors based on retroviruses, vectors with site specific recombination sequences, or by homologous recombination. Generally, these vectors include control sequences operably linked to the nucleic acids encoding the peptides. By “control sequences” is meant nucleic acid sequences necessary for expression of the subject peptides in a particular host organism. Thus, control sequences include sequences required for transcription and translation of the nucleic acids, including, but not limited to, promoter sequences, enhancer or transcriptional activator sequences, ribosomal binding sites, transcriptional start and stop sequences; polyadenylation signals; etc.

A variety of promoters are useful in expressing the peptides of the present invention. The promoters may be constitutive, inducible, and/or cell specific and may comprise natural promoters, synthetic promoters (e.g. tTA tetracycline inducible promoters), or hybrids of various promoters. Promoters are chosen based on, among others, the cell or organism in which the proteins are to be expressed, the level of desired expression, and regulation of expression. Suitable promoters are bacterial promoters (e.g., pL I phage promoter, tac promoter, lac lac promoter, etc.); yeast based promoters (e.g., GAL4 promoter, alcohol dehydrogenase promoter, tryptophane synthase promoter, copper inducible CUPI promoter, etc.), plant promoters (e.g., CaMV S35, nopoline synthase promoter, tobacco mosaic virus promoter, etc), insect promoters (e.g., Autographa nuclear polyhedrosis virus, Aedes DNV viral p& and p61, hsp70, etc.), and promoters for expression mammalian cells (e.g., ubiquitin gene promoter, ribosomal gene promoter, β-globin promoter, thymidine kinase promoter, heat shock protein promoters, and ribosomal gene promoters, etc.), and particularly viral promoters, such as cytomegalovirus (CMV) promoter, simian virus (SV40) promoter, and retroviral promoters.

By “operably linked” herein is meant that a nucleic acid is placed into a functional relationship with another nucleic acid. In the present context, operably linked means that the control sequences are positioned relative to the nucleic acid sequence encoding the subject peptides in such a manner that expression of the encoded peptide occurs. The vectors may comprise plasmids or comprise viral vectors, for example retroviral vectors, which are useful delivery systems if the cells are dividing cells, or lentiviral and adenoviral vectors if the cells are non-dividing cells. Particularly preferred are self-inactivating retroviral vectors (SIN vectors), which have inactivated viral promoters at the 3′-LTR, thereby permiting control of expression of heterologous genes by use of non-viral promoters inserted into the viral vector (see for example, Hoffman et al. Proc. Natl. Acad. Sci. USA 93: 5185 (1996). As will be appreciated by those in the art, modifications of the system by pseudotyping allows use of retroviral vectors for all eukaryotic cells, particularly for higher eukaryotes (Morgan, R. A. et al. J. Virol. 67: 4712-21 (1993); Yang, Y. et al. Hum. Gene Ther. 6: 1203-13 (1995)).

In addition, the expression vectors also contain a selectable marker gene to allow selection of transformed host cells. Generally, the selection will confer a detectable phenotypes that enriches for cells containing the expression vector and further permits differentiation between cells that express and do not express the selection gene. Selection genes are well known in the art and will vary with the host cell used. Suitable selection genes included genes that render the cell resistant to a drug, genes that permit growth in nutritionally deficient media, and reporter genes (e.g. β-galactosidase, fluorescent proteins, glucouronidase, etc.), all of which are well known in the art and available to the skilled artisan.

There are a variety of techniques available for introducing nucleic acids into viable cells. By “introduced” into herein is meant that the nucleic acid enters the cells in a manner suitable for subsequent expression of the nucleic acid. Techniques for introducing the nucleic acids will vary depending on whether the nucleic acid is transferred in vitro into cultured cells or in vivo into the cells of the intended host organism and the type of host organism. Exemplary for introducing the nucleic acids in vitro include the use of liposomes, Lipofectin®, electroporation, microinjection, cell fusion, DEAE dextran, calcium phosphate prepcipitation, and bioloistic particle bombardment. Techniques for transfer in vivo include direct introduction of the nucleic acid, use of viral vectors, typically retroviral vectors, and liposome mediated transfection, such as viral coated liposome mediated transfection. The nucleic acids expressing the peptides of the present invention may exist transiently or stably in the cytoplasm or stably integrate into the chromosome of the host (i.e., through use of standard regulatory sequences, selection markers, etc.). Suitable selection genes and marker genes are used in the expression vectors of the present invention.

In some situations, it is desirable to include an agent that targets the target cells or tissues, such as an antibody specific for a cell surface protein or the target cell, a ligand for a receptor on the target cell, a lipid component on the cell membrane, or a carbohydrate on the cell surface. If liposomes are employed, proteins that bind a cell surface protein which is endocytosed may be used for targeting and/or facilitating uptake. These include as non-limiting examples, capsid proteins or fragments thereof tropic for a particular cell types, antibodies for proteins which undergo internalization (see Wu et al. J. Biol. Chem. 262: 4429-4432 (1987); Wagner et al. Proc. Natl. Acad. Sci. USA 87: 3410-3414 (1990)), and proteins that direct localization (e.g., antibody to transferrin receptor for targeting to brain) or enhance in vivo half-life.

Expression is done in a wide range of host cells that span prokaryotes and eukaryotes, including bacteria, yeast, plants, insects, and animals. The immunomodulatory peptides of the present invention may be expressed in, among others, E. coli., Saccharomyces cerevisiae, Saccharomyces pombe, Tobacco or Arabidopsis plants, insect Schneider cells, and mammalian cells, such as COS, CHO, HeLa, and the like, either intracellularly or in a secreted form by fusing the peptides to an appropriate signal peptide. Secretion from the host cell may be done by fusing the DNA encoding the peptide and a DNA encoding a signal peptide. Secretory signals are well known in the art for bacteria, yeast, insects, plants, and mammalian systems. Nucleic acids expressing the opeptides may be inserted into cells, for example stem cells for tissue expression or bacteria for gut expression, and the cells transplanted into the host to provide an in vivo source of the peptides.

If desired, various groups are introduced into the peptide during synthesis or during expression, which allows for linking to other molecules or to a surface. Thus, cysteines can be used to make thioethers or cyclic peptides, histidines for linking to a metal ion complex, carboxyl groups for forming amides or esters, amino groups for forming amides, and the like. When cysteine residues are introduced for cyclizing the peptide, formation of disulfide bonds are conducted in the presence of mild oxidizing agents. Chemical oxidants may be used, or the cysteine bearing peptides are exposed to oxygen to form the linkages, typically in a suitable solution such as a aqueous buffer containing DMSO. As described above, lipids may be attached either chemically or by use of appropriate lipidation sequences in the expressed peptide.

For conjugating various molecules to the peptides of the present invention, functional groups on the peptides and the other molecule are reacted in presence of an appropriate conjugating (e.g., crosslinking) agent. The type of conjugating or crosslinking agent used will depend on the functional groups, such as primary amines, sulfhydryls, carbonyls, carbohydrates and carboxylic acids being used. Agents may be fixatives and crosslinking agents, which may be homobifunctional, heterobifunctional, or trifunctional crosslinking agents (Pierce Endogen, Chicago, Ill.). Commonly used fixatives and crosslinking agents include formaldehyde, glutaraldehyde, 1,1-bis(diazoacetyl)-2-phenylethane, N-hydroxysuccinimide esters, dissuccimidyl esters, maleimides (e.g., bis-N-maleimido-1-8-octane), and carbodiimides (e.g., N-ethyl-N′-(3-dimethylaminopropyl)-carbodiimide; dicyclohexylcarbodiimide. Spacer molecules comprising alkyl or substituted alkyl chains with lengths of 2-20 carbons may be used to separate conjugates. Preferably, reactive functional groups on the peptide not selected for modification are protected prior to coupling of the peptide to other reactive molecules to limit undesired side reactions. By “protecting group” as used herein is a molecule bound to a specific functional group which is selectively removable to reexpose the functional group (see Greene, T. W. and Wuts, P. G. M. Protective Groups in Organic Synthesis (3rd ed.), John Wiley & Sons, Inc., New York, 1999). The peptides may be synthesized with protected amino acid precursors or reacted with protecting groups following synthesis but before reacting with crosslinking agent. Conjugations may also be indirect, for example by attaching a biotin moiety, which can be contacted with a compound or molecule which is coupled to streptavidin or avidin.

For peptides that have reduced activity in the conjugated form, the linkage between the peptides and the conjugated compound is chosen to be sufficiently labile to result in cleavage under desired conditions, for example after transport to desired cells or tissues. Biologically labile covalent bonds, e.g., imimo bonds and esters, are well known in the art (see U.S. Pat. No. 5,108,921, hereby incorporated by reference). These modifications permit administration of the peptides in potentially a less active form, which is then activated by cleavage of the labile bond.

In a preferred embodiment, the immunomodulatory peptides of the present invention may be purified or isolated after synthesis or expression. By “purified” or “isolated” is meant free from the environment in which the peptide is synthesized or expressed and in a form where it can be practically used. Thus purified or isolated is meant that the peptide or its derivative is substantially pure, i.e., more than 90% pure, preferably more than 95% pure, and preferably more than 99% pure. The peptides and derivatives thereof may be purified and isolated by way known to those skilled in the art, depending on other components present in the sample. Standard purification methods include electrophoretic, immunological, and chromatographic techniques, including ion exchange, hydrophobic, affinity, size exclusion, reverse phase HPLC, and chromatofocusing. The proteins may also be purified by selective solubility, for instance in the presence of salts or organic solvents. The degree of purification necessary will vary depending on use of the subject peptides. Thus, in some instances no purification will be necessary.

For the most part, the compositions used will comprise at least 20% by weight of the desired product, more usually at least about 75% by weight, preferably at least about 95% by weight, and usually at least about 99.5% by weight, relative to contaminants related to the method of product preparation, the purification procedure, and its intended use, for example with a pharmaceutical carrier for the purposes of therapeutic treatment. Usually, the percentages will be based upon total protein.

The subject peptides find use in treating gastrointestinal toxicity and dysfunction in patients undergoing cytoablative therapy, e.g., chemotherapy and radiation therapy. In addition, these peptides enable the clinical oncologist to administer an increased maximum tolerated dose of the cytoablative agent, thereby providing significant improvements in tumor response and life expectancy.

Additional Therapeutic Agents

Additional therapeutic or pharmaceutically active agents may also be advantageously used in combination with the above compositions, including corticosteroids (e.g., prednisone, methylprednisolone, dexamethasone, etc.); immunomodulators (e.g., interferon, including interferon—b1a, interferon-b1a); immune suppressants (e.g., azathioprine, 6-mercaptopurine, cyclosporin); anti-inflammatory compounds, including, but not limited to, non-steroidal anti-inflammatory compounds (e.g., sulfasalzine, aminosalicylates, celecoxib, lipoxins, etc.); anti-diarrheal agents such as loperamide, hydroxyurea; and thalidomide, which is known to increase IL-2 and IL-12 levels.

As will be appreciated by those skilled in the art, in certain circumstances where the gastrointestinal toxicity and dysfunction is further complicated by pathogen infection, the peptides of the present invention may be used with drugs directed against eliminating or killing the pathogen. These include antibiotics, anti-fungal agents, anti-protozoan agents, and anti-viral agents, as is well known in the art. These drugs may be used prior to, concomitantly with, or subsequent to treatment with the peptides described herein.

The present invention may also be used in combination of anti-inflammatory cytokines, growth factors, or leukocyte migration inhibitory compounds. Useful cytokines include, but are not limited to, IL-4, IL-10, IL-11, and IL-13, particularly IL-4 and IL-10, which are known to suppress production of inflammatory cytokines and to be involved in restoring the immune system. Growth factors include transforming growth factor-β (TGF-β) and GM-CSF. These cytokines and growth factors may be administered as purified proteins—obtained naturally or from recombinant sources—or administered in the form of nucleic acids that express these peptides, particularly as fusion proteins. Leukocyte migration inhibitory compounds, include, among others, antibodies directed against adhesion molecules and their cognate receptors involved in cell adhesion, particularly leukocyte adhesion to endothelial cells, such as for E-, L-, and P-selectins; vascular cell adhesion molecule-1 (VCAM-1); mucosal addressin cell adhesion molecule, (MAdCAM-1); and intercellular adhesion molecule-1 (ICAM-1); and their cognate receptors, such as α₄β₁ and α₄β₇.

In another preferred embodiment, the immunomodulatory peptides are further combined with other inhibitors of pro-inflammatory cytokine activity or agents that reduce synthesis of these cytokines. These include agents that block cytokine function, such as antibodies to IL-5, IL-6, IL-8, IL-18, IL-23, TNF-α, and IFN-γ, and antibodies to their cognate receptors; and cytokine receptor antagonists (see for example, U.S. Pat. No. 6,436,927). In addition, blocking agents include soluble receptors proteins, for instance receptors fused to IgC domains, that bind to cytokines to reduce activation of CD4⁺T-cells, macrophages, and granulocytes involved in progression of the inflammatory reaction.

Pharmaceutical Formulations

The subject compositions, either alone or in combination, may be used in vitro, ex vivo, and in vivo depending on the particular application. In accordance, the present invention provides for administering a pharmaceutical composition comprising a pharmaceutically acceptable carrier and a pharmacologically effective amount of one or more of the subject peptides, or suitable salts thereof. The pharmaceutical composition may be formulated as powders, granules, solutions, suspensions, aerosols, solids, pills, tablets, capsules, gels, topical cremes, suppositories, transdermal patches, etc.

As indicated above, pharmaceutically acceptable salts of the peptides is intended to include any art recognized pharmaceutically acceptable salts including organic and inorganic acids and/or bases. Examples of salts include sodium, potassium, lithium, ammonium, calcium, as well as primary, secondary, and tertiary amines, esters of lower hydrocarbons, such as methyl, ethyl, and propyl. Other salts include organic acids, such as acetic acid, propionic acid, pyruvic acid, maleic acid, succinic acid, tartaric acid, citric acid, benzoic acid, cinnamic acid, salicylic acid, etc.

As used herein, “pharmaceutically acceptable carrier” comprises any of standard pharmaceutically accepted carriers known to those of ordinary skill in the art in formulating pharmaceutical compositions. Thus, the subject peptides, by themselves, such as being present as pharmaceutically acceptable salts, or as conjugates, or nucleic acid vehicles encoding such peptides, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients. The pharmaceutical compositions also contain anti-retroviral agents when such agents are part of the compositions. Additionally, the formulations may include bactericidal agents, stabilizers, buffers, emulsifiers, preservatives, sweetening agents, lubricants, or the like. If administration is by oral route, the oligopeptides may be protected from degradation by using a suitable enteric coating, or by other suitable protective means, for example internment in a polymer matrix such as microparticles or pH sensitive hydrogels.

Suitable formulations may be found in, among others, Remington's Pharmaceutical Sciences, 17^{th} edition, Mack Publishing Co., Philadelphia, Pa., 1985 and Handbook of Pharmaceutical Excipients, 3rd Ed, Kibbe, A. H. ed., Washington DC, American Pharmaceutical Association, 2000; hereby incorporated by reference in their entirety. The pharmaceutical compositions described herein can be made in a manner well known to those skilled in the art (e.g., by means conventional in the art, including mixing, dissolving, granulating, levigating, emulsifying, encapsulating, entrapping or lyophilizing processes).

Additionally, the peptides, either alone or with other agents including chemotherapeutic agents may also be introduced or encapsulated into the lumen of liposomes for delivery and for extending life time of the peptide formulations ex vivo or in vivo. As known in the art, liposomes can be categorized into various types: multilamellar (MLV), stable plurilamellar (SPLV), small unilamellar (SUV) or large unilamellar (LUV) vesicles. Liposomes can be prepared from various lipid compounds, which may be synthetic or naturally occurring, including phosphatidyl ethers and esters, such as phosphotidylserine, phosphotidylcholine, phosphatidyl ethanolamine, phosphatidylinositol, dimyristoylphosphatidylcholine; steroids such as cholesterol; cerebrosides; sphingomyelin; glycerolipids; and other lipids (see for example, U.S. Pat. No. 5,833,948).

Cationic lipids are also suitable for forming liposomes. Generally, the cationic lipids have an net positive charge and have a lipophilic portion, such as a sterol or an acyl or diacyl side chain. Preferably, the head group is positively charged. Typical cationic lipids include 1,2-dioleyloxy-3-(trimethylamino)propane; N-[1-(2,3,-ditetradecycloxy)propyl]-N,N-dimethyl-N-N-hydroxyethylammonium bromide; N-[1-(2,3-dioleyloxy)propyl]-N,N-dimethyl-N-hydroxy ethylammonium bromide; N-[1-(2,3-dioleyloxy) propyl]-N,N,N-trimethylammonium chloride; 3-[N-(N′,N′-dimethylaminoethane) carbamoyl] cholesterol; and dimethyidioctadecylammonium.

Of particular interest are fusogenic liposomes, which are characterized by their ability to fuse with a cell membrane upon appropriate change in physiological condition or by presence of fusogenic component, particularly a fusogenic peptide or protein. In one aspect, the fusogenic liposomes are pH and temperature sensitive in that fusion with a cell membrane is affected by change in temperature and/or pH (see for example, U.S. Pat. Nos. 4,789,633 and 4,873,089). Generally, pH sensitive liposomes are acid sensitive. Thus, fusion is enhanced in physiological environments where the pH is mildly acidic, for example the environment of a lysosome, endosome and inflammatory tissues. This property allows direct release of the liposome contents into the intracellular environment following endocytosis of liposomes (see Mizoue, T. Int. J. Pharm. 237: 129-137 (2002)).

Another form of fusogenic liposomes comprise liposomes that contain a fusion enhancing agent. That is, when incorporated into the liposome or attached to the lipids, the agents enhance fusion of the liposome with other cellular membranes, thus resulting in delivery of the liposome contents into the cell. The agents may be fusion enhancing peptides or proteins, including hemaggulutinin HA2 of influenza virus (Schoen, P. Gene Ther. 6: 823-832 (1999)); Sendai virus envelope glycoproteins (Mizuguchi, H. Biochem. Biophys. Res. Commun. 218: 402-407 (1996)); vesicular stomatitis virus envelope glycoproteins (VSV-G) glycoprotein (Abe, A. et al. J Virol 72: 6159-63 (1998)); peptide segments or mimics of fusion enhancing proteins; and synthetic fusion enhancing peptides (Kono, K. et al. Biochim. Biophys. Acta. 1164: 81-90 (1993); Pecheur, E. I. Biochemistry 37: 2361-71 (1998); U.S. Pat. No. 6,372,720).

Liposomes also include vesicles derivatized with a hydrophilic polymer, as provided in U.S. Pat. Nos. 5,013,556 and 5,395,619, hereby incorporated by reference, (see also, Kono, K. et al. J. Controlled Release 68: 225-35 (2000); Zalipsky, S. et al. Bioconjug. Chem. 6: 705-708 (1995)) to extend the circulation lifetime in vivo. Hydrophilic polymers for coating or derivation of the liposomes include polyethylene glycol, polyvinylpyrrolidone, polyvinylmethyl ether, polyaspartamide, hydroxymethyl cellulose, hydroxyethyl cellulose, and the like. In addition, as described above, attaching proteins that bind a cell surface protein which is endocytosed, e.g., capsid proteins or fragments thereof tropic for a particular cell types and antibodies for cell surface proteins which undergo internalization (see Wu et al, supra; Wagner et al., supra), may be used for targeting and/or facilitating uptake of the liposomes to specific cells or tissues.

Liposomes are prepared by ways well known in the art (see for example, Szoka, F. et al. Ann. Rev. Biophys. Bioeng. 9: 467-508 (1980)). One typical method is the lipid film hydration technique in which lipid components are mixed in an organic solvent followed by evaporation of the solvent to generate a lipid film. Hydration of the film in aqueous buffer solution, preferably containing the subject peptide or nucleic acid, results in an emulsion, which is sonicated or extruded to reduce the size and polydispersity. Other methods include reverse-phase evaporation (see Pidgeon, C. et al. Biochemistry 26: 17-29 (1987); Duzgunes, N. et al. Biochim. Biophys. Acta. 732: 289-99 (1983)), freezing and thawing of phospholipid mixtures, and ether infusion.

In another preferred embodiment, the carriers are in the form of microparticles, microcapsules, micropheres and nanoparticles, which may be biodegradable or non-biodegradable (see for example, Microencapsulates: Methods and Industrial Applications, Drugs and Phamaceutical Sciences, Vol 73, Benita, S. ed, Marcel Dekker Inc., New York, 1996; incorporated by reference). As used herein, microparticles, microspheres, microcapsules and nanoparticles mean a particle, which is typically a solid, containing the substance to be delivered. The substance is within the core of the particle or attached to the particle's polymer network. Generally, the difference between microparticles (or microcapsules or microspheres) and nanoparticles is one of size. As used herein, microparticles have a particle size range of about 1 to about >1000 microns. Nanoparticles have a particle size range of about 10 to about 1000 nm.

A variety of materials are useful for making microparticles. Non-biodegradable microcapsules and microparticles include, but not limited to, those made of polysulfones, poly(acrylonitrile-co-vinyl chloride), ethylene-vinyl acetate, hydroxyethylmethacrylate-methyl-methacrylate copolymers. These are useful for implantation purposes where the encapsulated peptide diffuses out from the capsules. In another aspect, the microcapsules and microparticles are based on biodegradable polymers, preferably those that display low toxicity and are well tolerated by the immune system. These include protein based microcapsulates and microparticles made from fibrin, casein, serum albumin, collagen, gelatin, lecithin, chitosan, alginate or poly-amino acids such as poly-lysine. Biodegradable synthetic polymers for encapsulating may comprise polymers such as polylactide (PLA), polyglycolide (PGA), poly(lactide-co-glycolide) (PLGA), poly(caprolactone), polydioxanone trimethylene carbonate, polyhybroxyalkonates (e.g., poly(β-hydroxybutyrate)), poly(γ-ethyl glutamate), poly(DTH iminocarbony (bisphenol A iminocarbonate), poly (ortho ester), and polycyanoacrylate. Various methods for making microparticles containing the subject compositions are well known in the art, including solvent removal process (see for example, U.S. Pat. No. 4,389,330); emulsification and evaporation (Maysinger, D. et al. Exp. Neuro. 141: 47-56 (1996); Jeffrey, H. et al. Pharm. Res. 10: 362-68 (1993)), spray drying, and extrusion methods.

Another type of carrier is nanoparticles, which are generally suitable for intravenous administrations. Submicron and nanoparticles are generally made from amphiphilic diblock, triblock, or multiblock copolymers as is known in the art. Polymers useful in forming nanoparticles include, but are limited to, poly(lactic acid) (PLA; see Zambaux et al., J. Control Release 60: 179-188 (1999)), poly(lactide-co-glycolide), blends of poly(lactide-co-glycolide) and polycarprolactone, diblock polymer poly(l-leucine-block-I-glutamate), diblock and triblock poly(lactic acid) (PLA) and poly(ethylene oxide) (PEO) (see De Jaeghere, F. et al., Pharm. Dev. Technol. ;5: 473-83 (2000)), acrylates, arylamides, polystyrene, and the like. As described for microparticles, nanoparticles may be non-biodegradable or biodegradeable. Nanoparticles may be also be made from poly(alkylcyanoacrylate), for example poly(butylcyanoacrylate), in which the peptide is absorbed onto the nanoparticles and coated with surfactants (e.g., polysorbate 80). Methods for making nanoparticles are similar to those for making microparticles and include, among others, emulsion polymerization in continuous aqueous phase, emulsification-evaporation, solvent displacement, and emulsification-diffusion techniques (see Kreuter, J. Nano-particle Preparation and Applications, In Microcapsules and nanoparticles in medicine and pharmacy,” (M. Donbrow, ed.), pg. 125-148, CRC Press, Boca Rotan, Fla., 1991; incorporated by reference).

Hydrogels are also useful in delivering the subject agents into a host. Generally, hydrogels are crosslinked, hydrophilic polymer networks permeable to a wide variety of drug compounds, including peptides. Hydrogels have the advantage of selective trigger of polymer swelling, which results in controlled release of the entrapped drug compound. Depending on the composition of the polymer network, swelling and subsequent release may be triggered by a variety of stimuli, including pH, ionic strength, thermal, electrical, ultrasound, and enzyme activities. Non-limiting examples of polymers useful in hydrogel compositions include, among others, those formed from polymers of poly(lactide-co-glycolide), poly(N-isopropylacrylamide); poly(methacrylic acid-g-polyethylene glycol); polyacrylic acid and poly(oxypropylene-co-oxyethylene) glycol; and natural compounds such as chrondroitan sulfate, chitosan, gelatin, or mixtures of synthetic and natural polymers, for example chitosan-poly(ethylene oxide). The polymers are crosslinked reversibly or irreversibly to form gels embedded with the oligopeptides of the present invention (see for example, U.S. Pat. Nos. 6,451,346; 6,410,645; 6,432,440; 6,395,299; 6,361,797; 6,333,194; 6,297,337 Johnson, O. et al., Nature Med. 2: 795 (1996); incorporated by reference in their entirety).

In one preferred embodiment, the gel polymers are acrylic acid polymers, preferably carbomers (e.g., carboxypolymethylene), such as Carbopol (e.g., Carbopol 420-430, 475, 488, 493, 910, 934P, 974P, and the like; Brock et al., Pharmacotherapy 14: 430-437 (1994)), which are non-linear polymers of acrylic acid crosslinked with polyalkenyl polyether. Others types of carbomers include acrylic acids crosslinked with polyfunctional compounds, such as polyallysucrose. In addition to the advantage of hydrating and swelling to a gel, which entraps the subject compounds and limits their release, carbomer gels are mucoadhesive. The polymers adheres to the intestinal mucosal membrane, thus resulting in local delivery of the peptides (see Hutton et al. Clin. Sci. 78: 265-271 (1990); Pullan et al., Gut 34: 676-679 (1993), hereby incorporated by reference). In addition, these polymers have the added advantage of limiting intestinal protease activity.

The concentrations of the peptides or nucleic acid encoding therefore and the anti-retroviral agents will be determined empirically in accordance with conventional procedures for the particular purpose. Generally, for administering the peptides ex vivo or in vivo for therapeutic purposes, the subject formulations are given at a pharmacologically effective dose. By “pharmacologically effective amount” or “pharmacologically effective dose” is an amount sufficient to produce the desired physiological effect or amount capable of achieving the desired result, particularly for treating the disorder or disease condition, including reducing or eliminating one or more symptoms of the disorder or disease, in this case the gastrointestinal toxicities and dysfunction outlined herein.

The amount administered to the host will vary depending upon what is being administered, the purpose of the administration, such as prophylaxis or therapy, the state of the host, the manner of administration, the number of administrations, interval between administrations, and the like. These can be determined empirically by those skilled in the art and may be adjusted for the extent of the therapeutic response. Factors to consider in determining an appropriate dose include, but is not limited to, size and weight of the subject, the age and sex of the subject, the severity of the symptom, the stage of the disease, method of delivery of the agent, half-life of the agents, and efficacy of the agents. Stage of the disease to consider include whether the disease is acute or chronic, relapsing or remitting phase, and the progressiveness of the disease. Determining the dosages and times of administration for a therapeutically effective amount are well within the skill of the ordinary person in the art. In the context of the present invention, the gastrointestinal toxicities associated with cytoablative therapies often present themselves in a regular and recurring fashion after administration of each round of the cytoablative therapy, and thus prophylactic administration of the subject peptides is also possible in view of the predictable nature of the associated toxicities.

For any compounds used in the present invention, the therapeutically effective dose and the maximum tolerated dose are readily determined by methods well known in the art. For example, an initial effective dose can be estimated initially from cell culture assays. An indicator of inflammatory response may be used, such as expression levels of pro-inflammatory cytokines, or inhibition of CTL activity. From this, the LC₅₀ (i.e., dose lethal to about 50% of cells in the cell culture) and the IC₅₀ (i.e. inhibitory dose) can be determined by the cell culture assays. A dose can then be formulated in animal models to generate a circulating concentration or tissue concentration, and the maximum tolerated dose determined as exemplified in the examples provided herein.

As noted, the toxicity and therapeutic efficacy are generally determined by cell culture assays and/or experimental animals, typically by determining a LD₅₀ (lethal dose to 50% of the test population) and ED₅₀ (therapeutically effectiveness in 50% of the test population). The dose ratio of toxicity and therapeutic effectiveness is the therapeutic index. Preferred are compositions, individually or in combination, exhibiting high therapeutic indices. Determination of the effective amount is well within the skill of those in the art, particularly given the detailed disclosure provided herein. One skilled in the art knows how to calculate dosage amounts for a subject, particularly a mammal, and more particularly a human, based on dosage amounts determined in other animal models. Specific conversion factors for converting dosage amounts from one animal to another (e.g. from mouse to human) are well known in the art and are fully described, e.g., on the Food and Drug Administration web site at www.fda.gov/cder/cancer/animalframe.htm (in the oncology tools section), the disclosure of incorporated by reference

Generally, in the case where formulations are administered directly to a host, the present invention provides for a bolus or infusion of the subject composition that will administered in the range of about 0.1-50, more usually from about 1-25 mg/kg body weight of host. The amount will generally be adjusted depending upon the half-life of the peptide and anti-retroviral agent, where the half life will generally be at least one minute, more usually at least about 10 min, desirably in the range of about 10 min to 12 h. Short half-lives are acceptable, so long as efficacy can be achieved with individual dosages, continuous infusion, or repetitive dosages. Formulations for administration may be presented in unit a dosage form, e.g., in ampules, capsules, pills, or in multidose containers or injectables.

Dosages in the lower portion of the range and even lower dosages may be employed, where the peptide has an enhanced half-life or is provided as a depot, such as a slow release composition comprising particles, a polymer matrix which maintains the peptide over an extended period of time (e.g., a collagen matrix, carbomer, etc.), use of a pump which continuously infuses the peptide over an extended period of time with a substantially continuous rate, or the like. The host or subject may be any mammal including domestic animals, pets, laboratory animals, primates, particularly humans subjects.

In addition to administering the subject peptide compositions directly to a cell culture in vitro, to particular cells ex vivo, or to a mammalian host in vivo, nucleic acid molecules (DNA or RNA) encoding the subject peptides may also be administered thereto, thereby providing an effective source of the subject peptides for the application desired. As described above, nucleic acid molecules encoding the subject peptides may be cloned into any of a number of well known expression plasmids (see Sambrook et al., supra) and/or viral vectors, preferably adenoviral or retroviral vectors (see for example, Jacobs et al., J. Virol. 66:2086-2095 (1992), Lowenstein, Bio/Technology 12:1075-1079 (1994) and Berkner, Biotechniques 6:616-624 (1988)), under the transcriptional regulation of control sequences which function to promote expression of the nucleic acid in the appropriate environment. Such nucleic acid-based vehicles may be administered directly to the cells or tissues ex vivo (e.g., ex vivo viral infection of cells for transplant of peptide producing cells) or to a desired site in vivo, e.g. by injection, catheter, orally (e.g., hybrogels), and the like, or, in the case of viral-based vectors, by systemic administration. Tissue specific promoters may optionally be employed, assuring that the peptide of interest is expressed only in a particular tissue or cell type of choice. Methods for recombinantly preparing such nucleic acid-based vehicles are well known in the art, as are techniques for administering nucleic acid-based vehicles for peptide production.

For the purposes of this invention, the methods of administration is chosen depending on the condition being treated, the form of the subject compositions, and the pharmaceutical composition. Administration of the subject peptides can be done in a variety of ways, including, but not limited to, cutaneously, subcutaneously, intravenously, orally, topically, transdermally, intraperitoneally, intramuscularly, nasally, and rectally (e.g., colonic administration). For example, microparticle, microsphere, and microencapsulate formulations are useful for oral, intramuscular, or subcutaneous administrations. Liposomes and nanoparticles are additionally suitable for intravenous administrations. Administration of the pharmaceutical compositions may be through a single route or concurrently by several routes. For instance, oral administration can be accompanied by rectal or topical administration to the affected area. Alternatively, oral administration is used in conjunction with intravenous or parenteral injections.

In one preferred embodiment, the method of administration is by oral delivery, in the form of a powder, tablet, pill, or capsule. Pharmaceutical formulations for oral administration may be made by combining one or more peptide and anti-retroviral agent with suitable excipients, such as sugars (e.g., lactose, sucrose, mannitol, or sorbitol), cellulose (e.g., starch, methyl cellulose, hydroxylmethyl cellulose, carbonxymethyl cellulose, etc.), gelatin, glycine, saccharin, magnesium carbonate, calcium carbonate, polymers such as polyethylene glycol or polyvinylpyrrolidone, and the like. The pills, tablets, or capsules may have an enteric coating, which remains intact in the stomach but dissolves in the intestine. Various enteric coating are known in the art, a number of which are commercially available, including, but not limited to, methacrylic acid-methacrylic acid ester copolymers, polymer cellulose ether, cellulose acetate phathalate, polyvinyl acetate phthalate, hydroxypropyl methyl cellulose phthalate, and the like. Alternatively, oral formulations of the peptides are in prepared in a suitable diluent. Suitable diluents include various liquid form (e.g., syrups, slurries, suspensions, etc.) in aqueous diluents such as water, saline, phosphate buffered saline, aqueous ethanol, solutions of sugars (e.g. sucrose, mannitol, or sorbitol), glycerol, aqueous suspensions of gelatin, methyl cellulose, hydroxylmethyl cellulose, cyclodextrins, and the like. As used herein, diluent or aqueous solutions also include infant formula, given that cytoablative therapies may also be necessitated in infants and children. In some embodiments, lipohilic solvents are used, including oils, for instance vegetable oils, peanut oil, sesame oil, olive oil, corn oil, safflower oil, soybean oil, etc.); fatty acid esters, such as oleates, triglycerides, etc.; cholesterol derivatives, including cholesterol oleate, cholesterol linoleate, cholesterol myristilate, etc.; liposomes; and the like.

In another preferred embodiment, administration is done rectally. This may use formulations suitable for topical application in the form of salves, tinctures, cremes, or for application into the lumen of the intestine by use of compositions in the form of suppositories, enemas, foams, etc. Suppositories may contain conventional suppository bases such as cocoa butter, carbowaxes, polyethylene glycols, or glycerides, which are solid or semi-solid at room temperature but liquid at body temperature.

In yet another preferred embodiment, the administration is carried out cutaneously, subcutaneously, intraperitonealy, intramuscularly and intravenously. As discussed above, these are in the form of peptides dissolved or suspended in suitable aqueous medium, as discussed above. Additionally, the pharmaceutical compositions for injection may be prepared in lipophilic solvents, which include, but is not limited to, oils, such as vegetable oils, olive oil, peanut oil, palm oil soybean oil, safflower oil, etc; synthetic fatty acid esters, such as ethyl oleate or triglycerides; cholesterol derivatives, including cholesterol oleate, cholesterol linoleate, cholesterol myristilate, etc.; or liposomes, as described above. The compositions may be prepared directly in the lipophilic solvent or preferably, as oil/water emulsions, (see for example, Liu, F. et al. Pharm. Res. 12: 1060-1064 (1995); Prankerd, R. J. J. Parent. Sci. Tech. 44: 139-49 (1990); U.S. Pat. No. 5,651,991).

The delivery systems also include sustained release or long term delivery methods, which are well known to those skilled in the art. By “sustained release or” “long term release” as used herein is meant that the delivery system administers a pharmaceutically effective amount of subject compounds for more than a day, preferably more than a week, and most preferable at least about 30 days to 60 days, or longer. Long term release systems may comprise implantable solids or gels containing the subject peptide, such as biodegradable polymers described above; pumps, including peristaltic pumps and fluorocarbon propellant pumps; osmotic and mini-osmotic pumps; and the like. Peristaltic pumps deliver a set amount of drug with each activation of the pump, and the reservoir can be refilled, preferably percutaneously through a port. A controller sets the dosage and can also provides a readout on dosage delivered, dosage remaining, and frequency of delivery. Fluorocarbon propellant pumps utilize a fluorocarbon liquid to operate the pump. The fluorocarbon liquid exerts a vapor pressure above atmospheric pressure and compresses a chamber containing the drug to release the drug. Osmotic pumps (and mini-osmotic pumps) utilize osmotic pressure to release the drug at a constant rate. The drug is contained in an impermeable diaphragm, which is surrounded by the osmotic agent. A semipermeable membrane contains the osmotic agent, and the entire pump is housed in a casing. Diffusion of water through the semipermeable membrane squeezes the diaphragm holding the drug, forcing the drug into bloodstream, organ, or tissue. These and other such implants are particularly useful in treating a inflammatory disease condition, especially those manifesting recurring episodes or which are progressive in nature, by delivering the oligopeptides of the invention via systemic (e.g., intravenous or subcutaneous) or localized doses in a sustained, long term manner.

The present invention also encompasses the therapeutic combinations disclosed herein in the form of a kit or packaged formulation. A kit or packaged formulation as used herein includes one or more dosages of an immunomodulating peptide, and salts thereof, and optionally at least one additional therapeutic agent for combination therapy such as an anti-inflammatory or anti-diarrheal agent, in a container holding the dosages together with instructions for simultaneous or sequential administration to a patient. For example, the package may contain the peptides along with a pharmaceutical carrier combined in the form of a powder for mixing in an aqueous solution, which can be ingested by the afflicted subject. Another example of packaged drug is a preloaded pressure syringe, so that the compositions may be delivered colonically. The package or kit includes appropriate instructions, which encompasses diagrams, recordings (e.g., audio, video, compact disc), and computer programs providing directions for use of the combination therapy.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching.

All publications and patent applications mentioned in this specification are herein incorporated by reference to the same extent as if each individual publication or patent application was specifically and individually indicated to be incorporated by reference.

Experimental

As disclosed in U.S. patent applications Ser. No. 08/838,916 and U.S. Ser. No. 09/028,083 and in the relevant literature, including Grassy et al., parameters were defined based on known oligopeptides that had previously been found to have properties of inhibiting T cell activity (see, e.g., Buelow et al., supra). The conformational space necessary for immunosuppressive activity was computed according to the procedure described by Yasri et al., supra. Using these parameters (see Table I), which define compounds having known T cell inhibitory activity, new cytomodulating peptides were devised and tested, and were found to have activity equal to or surpassing known active compounds. The computer program used to predict and to devise the immunosuppressive activity of peptides and pseudopeptides was developed as follows:

1. Methodology

On the basis of an initial experimental data set made of peptides showing or not showing immunosuppressive activity, there was deduced:

i. A consensus sequence containing the amino acids required for the activity and allowing the development of new peptides or pseudopeptide libraries;

ii. A set of physicochemical and topological properties involved in the activity and converted into a set of constraints by the variable mapping technique (Grassy et al., J. of Molecular Graphics 13: 356-367 (1995)).

2. Variable Mapping

The method is based on physicochemical and conformational constraints, as deduced from the results of a training set of data.

Physicochemical Constraints

The method requires the determination of physicochemical constraints defined as ranges of properties for said biological activity. The computational method used for the determination of the set of constraints is named Variable Mapping and is described below.

The Variable Mapping Approach

This qualitative technique consists of an evaluation of the distribution (global or percent wise) of the active and inactive molecules as a function of the values of given parameters. The superposition of all graphs (activity-property) exhibits, for certain parameters, to the limiting values (low and/or higher) which are necessary for leading to an active compound. This graphical method gives a diagnosis of the qualitative non-linear dependencies between the activity and a molecular property. Regarding those properties involved in receptor ligand interactions, it has been clearly established that the existence of strict contingencies determining the adaptability to the receptor imply an embedding of certain structural and physicochemical properties. This method results in simple rules which can be used to predict the activity of unknown products. A graphical representation showing the number of successes relative to the number of violations of the rules allows one to compare the distributions with the activities for the whole set of molecules under study.

3. Physicochemical and Topological Parameters Used in the Definition of the Constraints Involved in the Immunosuppressive Activity of Peptides and Pseudopeptides.

Lipophilicity

Lipophilicity of peptides expressed as logP (where P is the partition coefficient of a named peptide between water and n-octanol). Molecular logP values can be computed by TSAR 2.31 using the atomic incremental logP values determined by Ghose et al., J. Chem. Inf. Comput. 29:163 (1989). As demonstrated by the analysis of the initial data set, the lipophilicity of an immunosuppressive peptide must be ≧−6.85

Topological Indices

Balaban index (Balaban, Chem. Phys. 89:399 (1982)):

The Balaban index computed for a connected molecular graph (H suppressed) is calculated as follows:

$$\frac{M}{μ + 1} {Σ ( {D}_{i} {D}_{j} )}^{- 0.5}$$

where M is the number of edges in the graph, μ is the cyclomatic number of the graph, i.e., the minimum number of edges which must be removed before G becomes acyclic, and Dᵢ=ΣDᵢⱼ (with j=1) is a distance matrix of the shortest path between the two vertices.

Molecular Volume:

The molecular volume is computed assuming standard Van der Waals radii for each element. This calculation is done on the extended conformation of the peptide.

Ellipsoidal Volume:

This volume is computed after determination of the three components of the inertia momentum of the molecule, assuming mean atomic masses for constituent atoms. This calculation is done on the extended conformation of the peptide.

Molar Refractivity:

Molar refractivity is computed using the atomic molar refractivity values determined by Ghose et al., supra.

Dipole Moment:

This parameter is computed on the extended conformation of the peptides. The total dipole moment for a molecule is expressed in Debye units:

μ=eΣrᵢqᵢ

where rᵢ is the distance of an atom i to the origin, qᵢ is the charge of the atom i. The charges on the atoms are computed using the Charge-2 method. (Abraham and Smith., J. Comput. Aided Mol. Design 3: 175-187 (1989))

Kier Chir V 4:

This index is one of the connectivity indexes developed by L. B. Kier. The Kier Chi V 4 computes in several steps (H included).

a. Determination and numbering of all the paths of length 4 on the molecular graph of the peptide.

b. Computation of each path of length 4 of the following quantities:

cₛ^{v}Π[(∂ⱼ^{v})]^{-0.5}

for j=1,4, where δᵢ=Zᵢ−hᵢ is defined for an atom as the difference between the total number of valence electrons Zᵢ and the number hᵢ of hydrogen atoms bonded to the atom i.

c. Summation of all these values concerning the entire set of subgraphs of length 4 on the graph

χ=Σ(cᵥ^{s})

Kier Kappa Alpha:

Kier Kappa Alpha 1 (Kα¹)

If A is the total number of atoms of the molecule (H included, Kα¹ is equal to:

$$\frac{( A + α ) {( A + α - 1 )}^{2}}{{( {P}_{1} + α )}^{2}}$$

with:

$${α}_{i} = \frac{{r}_{i}}{{rC}_{{sp}^{3}}} - 1$$

rᵢ is the covalent radius of the atom i and rCₛₚ³ the covalent radius of a carbon sp³, P₁ is the total number of paths of length=1 along the molecular graph of the peptide under study.

Kier Kappa Alpha 2 (Kα²)

If A is the total number of atoms of the molecule (H included), Kα² is equal to:

$$\frac{( A + α - 1 ) {( A + α - 2 )}^{2}}{{( {P}_{2} + α )}^{2}}$$

with:

$${α}_{i} = \frac{{r}_{i}}{{rC}_{{sp}^{3}}} - 1$$

rᵢ is the covalent radius of the atom i and rCₛₚ³ the covalent radius of a carbon sp³, P₂ is the total number of paths of length=2 along the molecular graph of the peptide under study.

Flexibility Phi:

Based upon the above formulas, the flexibility of a molecule can be defined as:

Phi=(Kα¹)(Kα²)/A

where A is the total number of atoms (H included).

Atoms and Groups Counts:

The number of the following atom types was also used as a constraint:

Total number of oxygen atoms of the peptide

Total number of nitrogen atoms of the peptide

The number of the following groups was also used as a constraint:

Total number of ethyl groups

Total number of hydroxyl groups

4. Values of the Constraints

Generation of Peptide or Pseudopeptide Libraries

Starting from the consensus sequence Arg-X-X-X-Arg-X-X-X-X-Tyr where X is an amino acid which is as defined above and in the earlier analogous formula, the physicochemical and topological parameters previously described were computed and whether these parameters were within the constraints defined by the initial training set. For example, starting from X=Leu, nLeu, Trp, Tyr, Gly or Val, a library of 279,936 molecules was generated and only 26 of them satisfied the required constraints.

The ranges of properties necessary to obtain a biological activity are summarized in the following Table I.

TABLE 1
Value ranges of physicochemical and structural parameters

| Property | Minimum | Maximum |
|---|---|---|
| LogP | −6.849 | −0.004 |
| Ellipsoidal Volume (Å³) | 5785.5 | 29460.00 |
| Molecular Volume (Å³) | 660.9 | 1050.4 |
| Molar refractivity | 221.30 | 359.3 |
| Kier Chi V4 | 3.325 | 5.342 |
| Kappa α₂ | 26.120 | 44.31 |
| Flexibility | 22.50 | 40.3 |
| Balaban Index | 2.846 | 6.701 |
| Total Dipole | 3.423 | 80.79 |
| Number of oxygen atoms | 10 | 15 |
| Number of nitrogen atoms | 8 | 20 |
| Number of ethyl groups | 0 | 1 |
| Number of hydroxyl groups | 1 | 3 |

5. Characterization of the Conformational Space Involved in the Immunosuppressive Activity of Peptides and Pseudopeptides.

Spatial Autocorrelation Vector of a 3D Structure:

The concept of autocorrelation description of a molecular structure was first introduced by Broto et al., Eur. J. Med. Chem. 19:66-70 (1984). This vector basically represents the discretized distance distribution derived from the interatomic distance matrix of a molecule. The first component of this vector (Aₒ) is equal to the number of atoms of the structures, the other components, A₁. . . Aₙ, are defined by the number of atom pairs which are separated by a distance within the range defined by a lower limit (n-1)Dᵢ, where n is the order of the bin of the vector and Dᵢ the distance increment. Similarly, it is possible to calculate the distribution of an atomic property P. In this case, the weighted autocorrelation component APₙ is obtained by the sum of the products of property values P on atoms i,j, having an interdistance belonging to the distance interval [(n-1)Dᵢ, nDᵢ]. The number of components of the vector is then defined by nₘₐₓ=(Dₘₐₓ/Dᵢ)+1, where Dₘₐₓ is the greatest interatomic distance in the structure.

The autocorrelation vector exhibits some useful features:

This vector achieves a substantial reduction of conformational data. An entire conformation is described by a limited set of n numerical values.

The vector is very easy to calculate on the basis of 3D coordinate data. Therefore, it is possible to compute and store this vector during molecular dynamics simulations, the reduction of the size of the storage involved in such a process, in comparison to the classical storage of a set of complete distance matrices, allows much longer simulations than usual.

The autocorrelation vector of a conformation is transitionally and rotationally invariant and is also independent of the atomic numbering of the molecule.

This vector is sensitive both to minor and major changes in conformation: the more the conformation is changed, the more the components of the vector are modified. The sensitivity depends on the distance increment chosen for calculations, but an increment from 0.5 Å or 1 Å (small molecules) to 5 Å (macromolecules) is a good choice for the usual simulations (Yasri et al., Protein Engineering 11:959-976 (1996)).

It is possible to analyze only a part of a structure or only a specific subset of atoms of this structure, e.g. Cₐ in proteins, N atoms, heavy atoms, etc. The vector is entirely defined by the knowledge of a structure, so that the comparison of different structures can be performed, using this vector without any reference.

Molecular Dynamics Analysis Using 3-D Autocorrelation Vectors

Applied to HLA-B2702.75-84 peptide (amino acid sequence Arg-Glu-Asn-Leu-Arg-Ile-Ala-Leu-Arg-Tyr) and on various active and inactive derivative peptides thereof, molecular dynamics simulations were performed using AMBER 4.1. The simulation of one nanosecond of dynamics generate a set of 10³ conformations (one conformation per picosecond). For each conformation the 3D autocorrelation vector was calculated using TSAR with a distance increment of 1 Å and the entire set of conformations was stored as 3D autocorrelation vectors versus time matrix (10³×n).

The aim of the work was to define the conformational space responsible for immunosuppressive activity, by comparison of the conformational spaces of active and inactive peptides using the methodology explained in the references cited in the Relevant Literature.

6. Statistical Analysis

Cluster Analysis

In order to compare different conformations, the distance matrix between all of these conformations in the hyperspace defined by the components of their unweighted 3D autocorrelation vectors was determined. The more the structure of two compounds are analogous, the shorter their distance. This method provides a quantification of the rigid molecular fit. Using the starting conformation as a reference, the numerical value of this distance is analogous to a root-mean-square deviation.

Principal Component Analysis (PCA)

PCA is a multidimensional statistical method for data analysis, suited for representing molecules in the hyperspace of their properties (molecular descriptors). PCA can be used to reduce a large number of descriptors to a smaller number of synthetic orthogonal variables issued from a linear combination of the original descriptors. This method retains the largest part of the total initial information. The original variables were normalized and the diagonalization of the covariance matrix was calculated using the classical Jacobi transform routine. The components of the 3K autocorrelogram vector provide a good description of the 3K structure of different conformations, but are awkward to handle because they contain too many data to get an easy visualization. PCA can reduce the dimensionality of the data to a 2D or 3D representation that contains as much of the original information as possible. Using PCA, the immunosuppressive peptides exhibit a well defined common conformational space. All the peptides able to reach these conformational specifications can exhibit an immunosuppressive activity.

Conformational Space Coordinates of Peptide bc 1 nL Bioactive Conformation

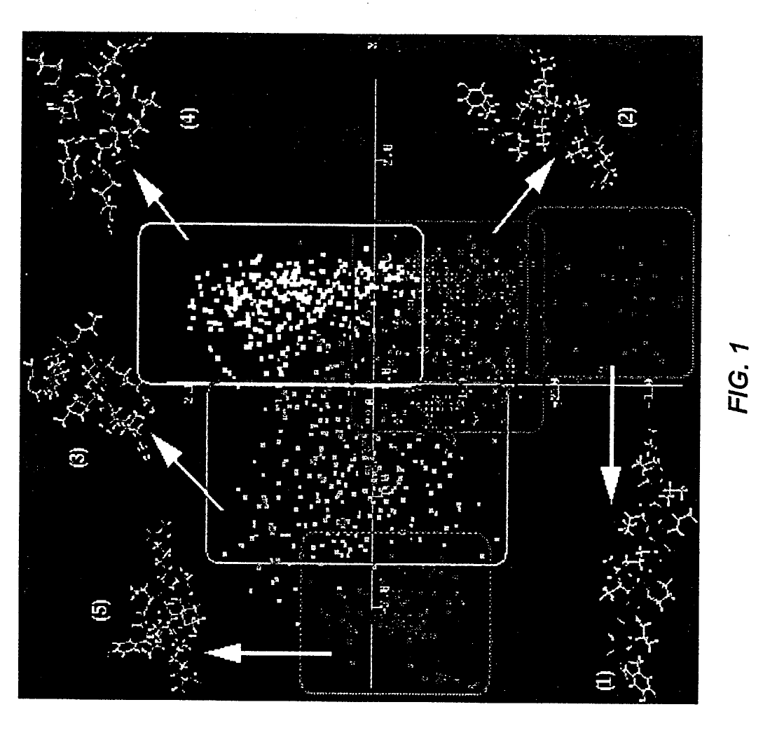

FIG. 1 shows the two-dimensional conformational space and related conformations of peptide bc 1 nL (RDP58), wherein the bc 1 nL peptide has the amino acid sequence Arg-nL-nL-nL-Arg-nL-nL-nL-Gly-Tyr and wherein “nL” is norleucine (see below). The structures drawn were obtained by applying cluster analysis method on the whole trajectory of peptide bc 1 nL.

Main Conformations of Peptide bc 1 nL

Structural properties of the main visited conformations of bc 1 nL (FIG. 1, (1), (2), (3), (4) and (5)) in its conformational space are summarized in Table II. These properties concern the coordinates in the three dimensional space defined by the three first principal components (PCA coordinates), and the radius of gyration (Rg).

TABLE 2
Spatial coordinates (PCA coordinates) and radius of gyration (Rg)
of dynamic conformations of peptide bc 1nL.

| | PCA Coordinates | | | |
|---|---|---|---|---|
| Conformations | PC1 | PC2 | PC3 | Rg |
| (1) | 0.785 | −2.816 | −0.531 | 9.92 |
| (2) | 0.382 | −0.899 | −0.164 | 7.99 |
| (3) | −0.811 | 0.345 | −0.481 | 6.93 |
| (4) | 0.741 | 0.950 | −1.092 | 6.76 |
| (5) | −2.096 | −0.296 | 0.770 | 6.67 |

Conformational Space of Active Peptides

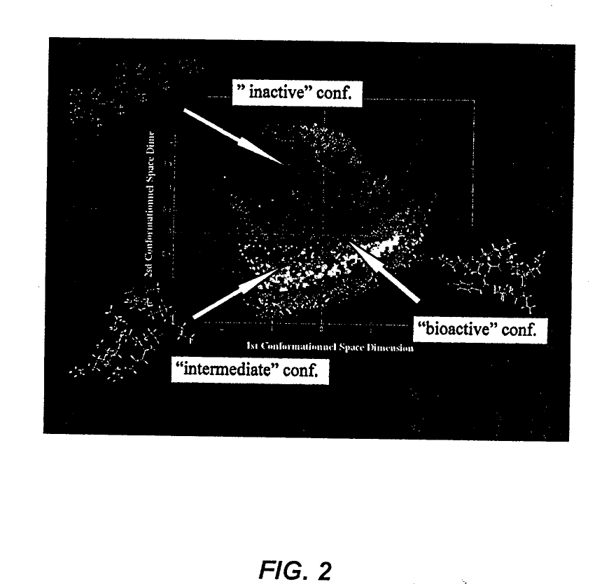

The trajectory of the D2 (amino acid sequence Arg-Val-Asn-Leu-Arg-Ile-Ala-Leu-Arg-Tyr) peptide has been described by the 3-D autocorrelation method and the data analyzed by principal component analysis. This provided a principal plan defined by the 2 first principal components which contain all the conformations visited during the trajectory. The D2 peptide trajectory was used as a trajectory reference and all the trajectories calculated were projected into its principal plan. (FIG. 2)

The immunosuppressive peptides exhibit a well defined common conformational space featuring the following points:

PCA Dimensions:

PC1:minimum=−2.0; maximum=2.0

PC2:minimum=−2.0; maximum=1.0

PC3:minimum=−1.0; maximum=1.0

The following peptides, defined as bc peptides, were devised:

TABLE 3

| bc # | | | | | | | | | | |
|---|---|---|---|---|---|---|---|---|---|---|
| 1 | Arg | Leu | Leu | Leu | Arg | Leu | Leu | Leu | Gly | Tyr |
| 2 | Arg | Val | Leu | Leu | Arg | Leu | Leu | Leu | Gly | Tyr |
| 3 | Arg | Ile | Leu | Leu | Arg | Leu | Leu | Leu | Gly | Tyr |
| 4 | Arg | Leu | Val | Leu | Arg | Leu | Leu | Leu | Gly | Tyr |
| 5 | Arg | Leu | Ile | Leu | Arg | Leu | Leu | Leu | Gly | Tyr |
| 6 | Arg | Leu | Leu | Val | Arg | Leu | Leu | Leu | Gly | Tyr |
| 7 | Arg | Leu | Leu | Ile | Arg | Leu | Leu | Leu | Gly | Tyr |
| 8 | Arg | Leu | Leu | Leu | Arg | Val | Leu | Leu | Gly | Tyr |
| 9 | Arg | Leu | Leu | Leu | Arg | Ile | Leu | Leu | Gly | Tyr |
| 10 | Arg | Leu | Leu | Leu | Arg | Leu | Val | Leu | Gly | Tyr |
| 11 | Arg | Leu | Leu | Leu | Arg | Leu | Ile | Leu | Gly | Tyr |
| 12 | Arg | Leu | Leu | Leu | Arg | Leu | Leu | Val | Gly | Tyr |
| 13 | Arg | Leu | Leu | Leu | Arg | Leu | Leu | Ile | Gly | Tyr |
| 14 | Arg | Trp | Leu | Leu | Arg | Leu | Leu | Leu | Gly | Tyr |
| 15 | Arg | Leu | Trp | Leu | Arg | Leu | Leu | Leu | Gly | Tyr |
| 16 | Arg | Leu | Leu | Trp | Arg | Leu | Leu | Leu | Gly | Tyr |
| 17 | Arg | Leu | Leu | Leu | Arg | Trp | Leu | Leu | Gly | Tyr |
| 18 | Arg | Leu | Leu | Leu | Arg | Leu | Trp | Leu | Gly | Tyr |
| 19 | Arg | Leu | Leu | Leu | Arg | Leu | Leu | Trp | Gly | Tyr |
| 20 | Arg | Tyr | Leu | Leu | Arg | Leu | Leu | Leu | Gly | Tyr |
| 21 | Arg | Leu | Tyr | Leu | Arg | Leu | Leu | Leu | Gly | Tyr |
| 22 | Arg | Leu | Leu | Tyr | Arg | Leu | Leu | Leu | Gly | Tyr |
| 23 | Arg | Leu | Leu | Leu | Arg | Tyr | Leu | Leu | Gly | Tyr |
| 24 | Arg | Leu | Leu | Leu | Arg | Leu | Tyr | Leu | Gly | Tyr |
| 25 | Arg | Leu | Leu | Leu | Arg | Leu | Leu | Tyr | Gly | Tyr |
| 1nL | Arg | nL | nL | nL | Arg | nL | nL | nL | Gly | Tyr |

nL = norleucine

### EXAMPLE 2

RDP58 Treatment Reduces Gastrointestinal Toxicity Induced by CPT-11

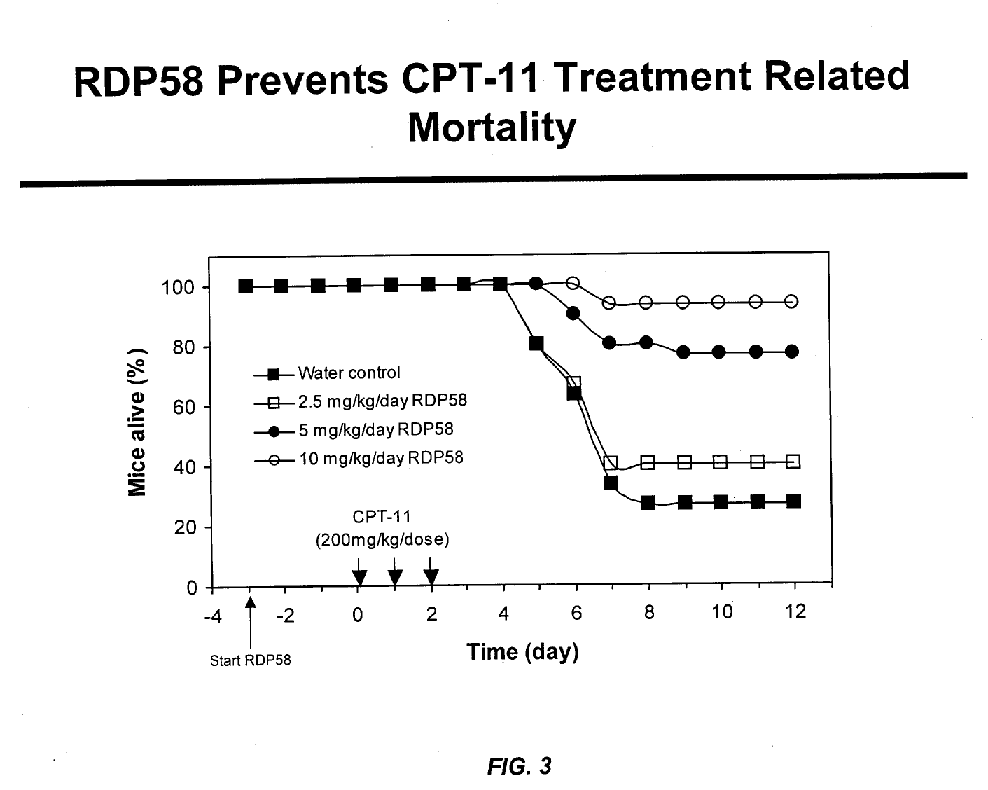

In cancer patients receiving CPT-11 therapy the development of diarrhea limits the administration of maximally effective chemotherapy. This study evaluated RDP58 as a potential protective agent to attenuate CPT-11-induced gastrointestinal toxicity and subsequent mortality in a murine tumor model. In a dose finding study, normal, non-tumor bearing mice were injected intraperitoneally with 200 mg/kg CPT-11 once a day for three consecutive days and concurrently given 0, 2.5, 5.0 or 10 mg/kg RDP58 orally. As shown in FIG. 3 and below in Table 4, RDP58 showed a dose dependent increase in survival twelve days after the first injection of CPT-11.

TABLE 4

| CPT-11 | RDP58 | Maximum | Toxicity (%) | |
|---|---|---|---|---|
| (mg/kg/dose) | (mg/kg/day) | weight loss | Diarrhea | Mortality |
| 200 | None | 29.4 ± 4.3 | 97 (29/30)^{b} | 73 (22/30) |
| 200 | 2.5 | 27.8 ± 3.7 | 67 (10/15)^{b} | 60 (9/15) |
| 200 | 5 | 25.6 ± 4.4^{a} | 40 (12/30)^{b} | 23 (7/30) |
| 200 | 10 | 24.6 ± 1.9^{a} | 33 (5/15)^{b} | 7 (1/15) |

a: p < 0.05, ANOVAb: p = 0.001, Cochran-Armitage trend test

Only one of fifteen mice died from CPT-11 treatment compared to 22 of 30 mice in the control group. A dose dependent decrease in appearance of diarrhea was also observed in mice receiving RDP58. Additionally, mice given 5.0 and 10.0 mg/kg RDP58 showed a reduction in the maximum total body weight lost compared to RDP58 untreated mice.

### EXAMPLE 3

RDP58 Treatment Reduces Gastrointestinal Toxicity Induced by 5-FU

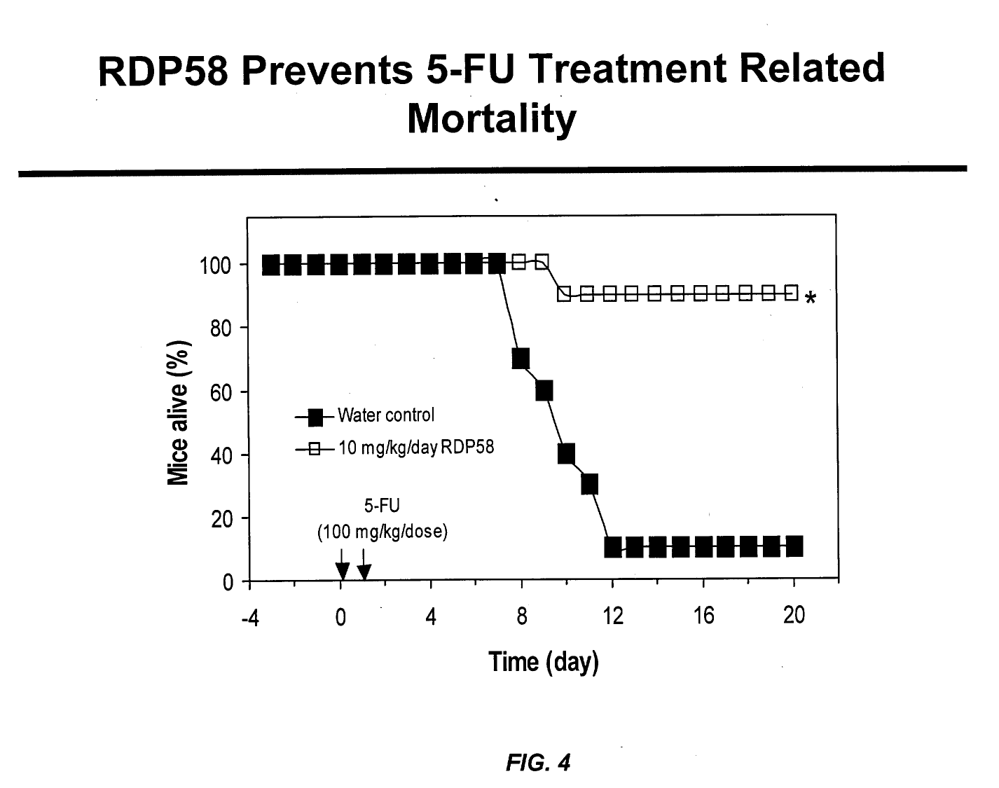

A second study was performed to determine whether RDP58 also limits 5-FU induced diarrhea. Normal, non-tumor bearing mice were treated with 100 mg/kg 5-FU daily for two consecutive days and were concurrently given 10 mg/kg RDP58 orally or water only. As shown in FIG. 4 and in Table 5 below, twelve days after 5-FU administration, RDP58 treated animals had a survival rate of 90% compared to 10% for untreated animals. All ten animals in the control group had diarrhea compared to only three of ten RDP58 treated animals. Additionally, a significant reduction in total body weight loss was observed in the group receiving RDP58.

TABLE 5

| 5-FU | RDP58 | Maximum | Toxicity (%) | |
|---|---|---|---|---|
| (mg/kg/dose) | (mg/kg/day) | weight loss | Diarrhea | Mortality |
| 100 | None | 34.7 ± 4.6 | 100(10/10) | 90(9/10) |
| 100 | 10 | 13.9 ± 3.8b | 30(3/10) | 10(1/10) |

b: p < 0.05, ANOV.

### EXAMPLE 4

RDP58 Reduces Treatment Related Mortality and Gastrointestinal Toxicity Without Decreasing CPT-11 Efficacy

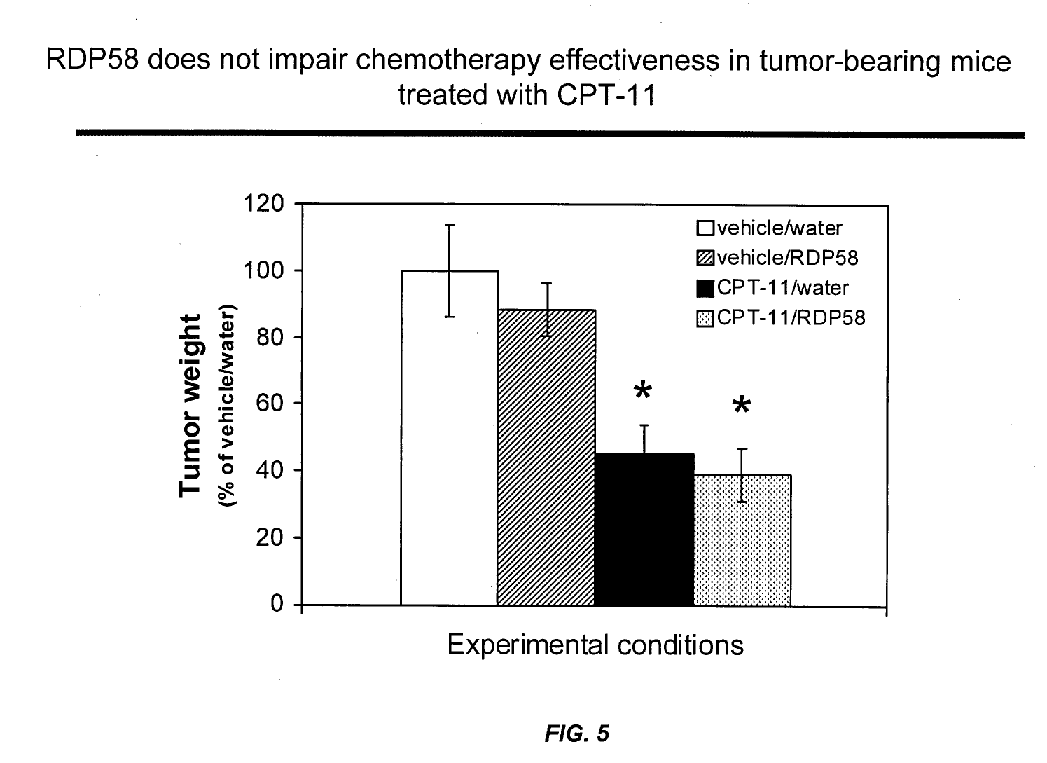

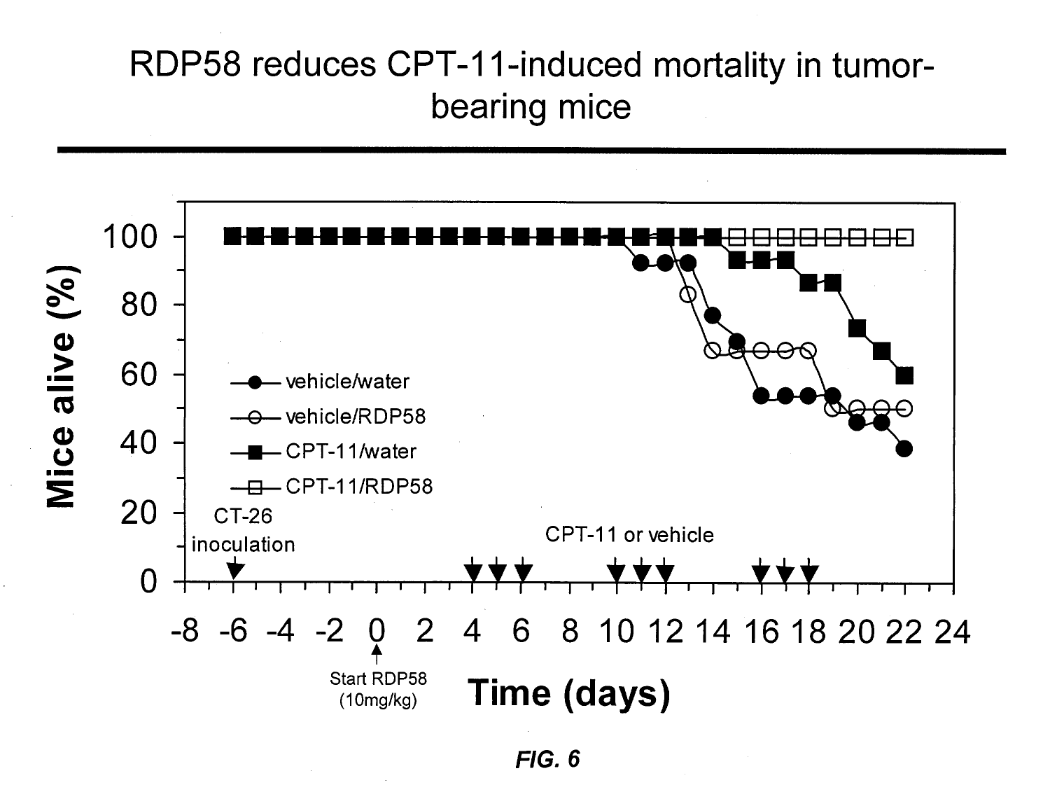

To determine whether RDP58 altered the efficacy of CPT-11 therapy, mice bearing CT-26 tumors were treated with three regimens of CPT-11 at 100 mg/kg qdx3 and given 5.0 mg/kg RDP58 orally. Both groups showed a similar reduction in tumor weight (FIG. 5, 55% decrease in RDP58 treated and 61% in RDP58 untreated). However, survival was significantly increased in groups given RPD58 (FIG. 6). No treatment related deaths were observed in the RDP58 group compared to 40% mortality in the control group.

### EXAMPLE 5

RDP58 Treatment Increases the Maximum Dose of CPT-11

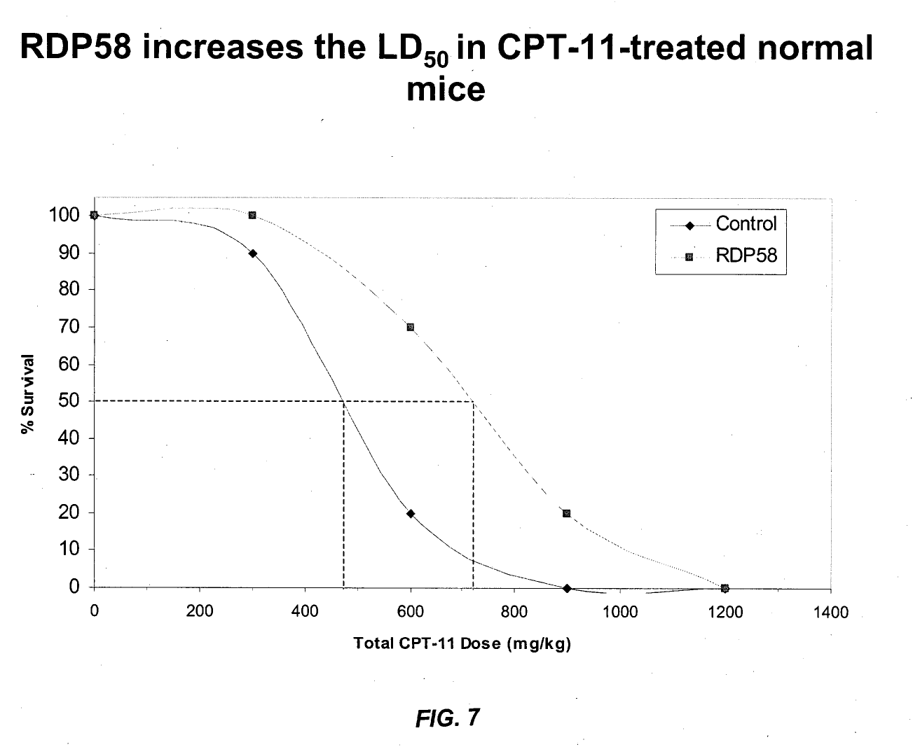

To determine whether concurrent oral RDP58 treatment can increase the maximum dose of CPT-11 without a concomitant increase in mortality, normal non-tumor bearing mice were injected qdx3 with 0, 100, 200, 300, or 400 mg/kg CPT-11 injected i.p. qdx3 on Days 0, 1 and 2. RDP58 was given orally at 10 mg/kg in the drinking water starting on Day 3. Mice were monitored for body weight loss, incidence of diarrhea and mortality until end of study on Day 12. The LD50 in the CPT-11 group was approximately 450 mg/kg/total dose. As shown in FIG. 7, administration of RDP58 increased the LD₅₀ to approximately 725 mg/kg/total dose, or by a factor of about 1.6×.

### EXAMPLE 6

RDP58 Treatment Enables an Increased Maximum Dose of CPT-11 with Lower Mortality and Improved Tumor Response

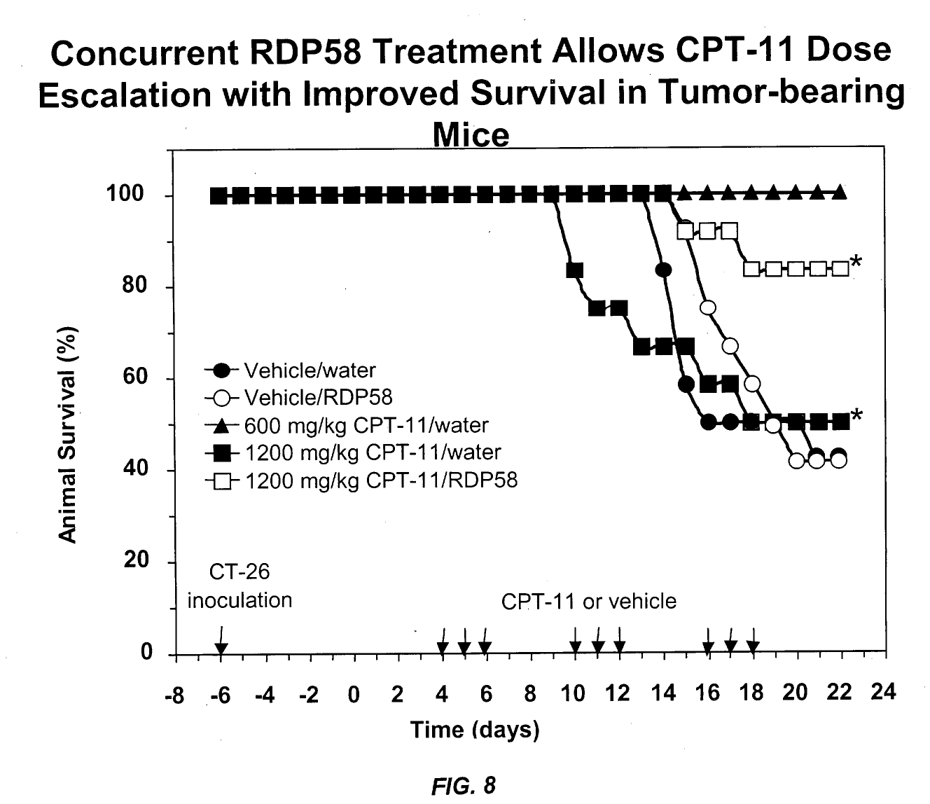

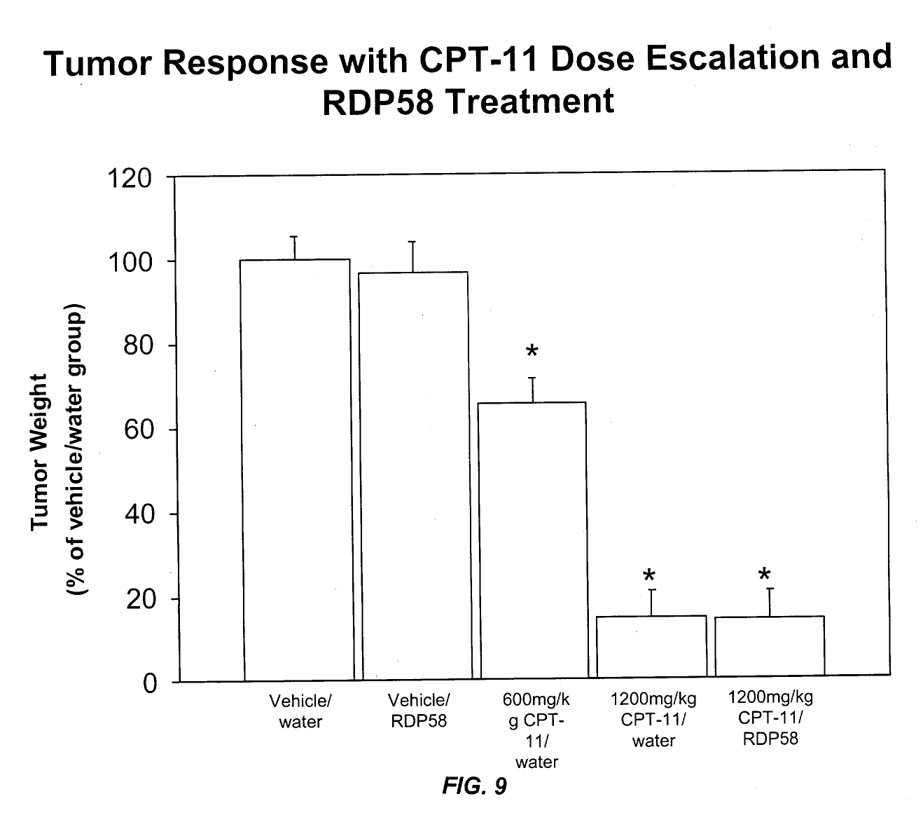

To determine if a reduction in GI toxicity and mortality with RDP58 treatment allows an increase in CPT-11 dosing, we compared treatment mortality and tumor response in tumor bearing. mice treated with the maximum tolerated dose (MTD) of Irinotecan (CPT-11), 2×MTD CPT-11, or 2×MTD plus 10 mg/ml RDP58. With our treatment schedule, we found the MTD to be 600 mg/kg CPT-11 (66.7 mg/kg/injection×9 injections over 12 days total). No animals in this treatment group died from the CPT-11, and tumors volumes decreased by about 40% compared to untreated controls (FIG. 9). Animals treated with 1200 mg/kg CPT-11 or 1200 mg/kg CPT-11+10 mg/ml RDP58 showed a decrease of greater than 80% in tumor volume. However, only 50% (6/12) of the animals in the 2×MTD CPT-11 group survived the complete treatment regimen whereas the CPT-11+RDP58 group had 83% (10/12) survive, as shown in FIG. 8. Although the mortality data is not statistically significant at the 95% confidence level (p=0.067, Log-Rank test), this data strongly supports the conclusion that escalating doses of RDP58 will enable an increase in MTD and thereby improve the anticancer response.

### EXAMPLE 7

RDP58 Treatment Reduces Proctitis Induced by Radiation Therapy

These studies evaluate RDP58 peptide administration in preventing or reducing radiation therapy-associated gastrointestinal distress. Study 1 finds the optimal dose and schedule of radiation to be used in subsequent efficacy studies. Study 2 assesses the protective effects of RDP58 in non-tumor bearing animals given an escalating dose of radiation. Study 3 assesses whether an increase in radiation dose to affect better tumor response can be achieved without a concomitant increase in toxicity. These studies determine whether oral RDP58 administration prevents or reduces treatment associated gastrointestinal toxicity and allows increased dosing for greater tumor responses in treatments for, e.g., metastatic colo-rectal, prostate and ovarian cancers.

Study 1—Radiation dose optimization: Normal healthy mice are given escalating doses of radiation to determine the dose of radiation that results in diarrhea in 90% of control animals. Animals are monitored daily for body weight loss, diarrhea, and morbidity. Animals losing >25% body weight or showing signs of severe distress are humanely sacrificed. The optimal dose and schedule that result in approximately 90% of treated animals having diarrhea is selected for the next studies. Alternatively, histological analysis of the intestine may be used to determine tissue damage.

Study 2—Assessment of RDP58 activity in normal, non-tumor bearing animals given escalating doses of radiation: The initial study uses 10 mg/kg RDP58 gavaged daily. This dose has been shown to reduce Irinotecan (CPT-11) and 5-Fluorouracil (5-FU) treatment associated mortality and diarrhea in mice and increases the LD50 of CPT-11 from about 465 mg/kg to 720 mg/kg total dose. Mice are monitored for body weight loss, incidence of diarrhea, and morbidity. If protection is demonstrated at the starting dose of radiation, a radiation dose escalating study will be performed to determine whether 10 mg/kg RDP58 allows increased radiation exposure while preventing or reducing GI toxicity and mortality. Histological analysis of intestinal epithelium is also performed of selected control and RDP58 treated animals to confirm clinical observations.

Study 3—Radiation dose escalation in tumor bearing animals: A syngeneic tumor model of ascites tumor is used. C57 BL/6 mice are injected intraperitoneally (i.p.) with EL-4 mouse thymoma or BALB/c mice are injected with CT-26 colo-rectal carcinoma cells and ascites tumors will be allowed to form. Alternatively, the lung metastatic 4T1 model may be used for studies using whole body irradiation. Mice start with oral 10 mg/kg RDP58 as described for Study 2. Mice with ascites are treated with 30 Gy; the dose of radiation and schedule is optimized to induce diarrhea in at least 90% of animals receiving without concurrent RDP58 administration. Mice are monitored for body weight loss, incidence of diarrhea, and morbidity. Thirty days after completion of radiation therapy, the mice are sacrificed and the tumor burden is determined by weight, histology, or assessed macroscopically for the volume of aspirated ascites. Histological analysis of gut tissue is also be performed of selected control and RDP58 treated animals.

The foregoing data demonstrate that immunomodulatory peptides can significantly ameliorate body weight loss, reduce diarrheal symptoms, and improve survival in CPT-11 and 5-FU administration. These data further suggest that RDP58 enables dose escalation of CPT-11 by limiting diarrhea—the major dose-limiting toxicity, and thereby improves long-term surivival.

All publications and patent applications mentioned in this specification are herein incorporated by reference to the same extent as if each individual publication or patent application was specifically and individually indicated to be incorporated by reference. The invention now being fully described, it will be apparent to one of ordinary skill in the art that many changes and modifications can be made thereto without departing from the spirit or scope of the appended claims.

What is claimed:

1. A method for reducing the gastrointestinal toxicities induced by cytoablative therapy, comprising administering to a patient undergoing cytoablative therapy a pharmaceutically effective amount of an immunomodulatory peptide, wherein said immunomodulatory peptide comprises an oligopetide comprising the amino acid sequence
B-X-X-X-B-X-X-X-J-Tyr
wherein
B is Lys or Arg;
X is a aliphatic or aromatic amino acid;
J is Gly, Lys or Arg,
wherein said amino acids are the naturally occurring L-isomers, their D-isomers, and norleucine.

2. A method according to claim 1, wherein for said oligopeptide
B is Arg;
X is an aliphatic non-polar amino acid of from 5 to 6 carbon atoms including norleucine, aromatic amino acid or the D-isomer thereof, there being at least 3 of the same aliphatic non-polar amino acid in said oligopeptide; and
J is Gly or Arg

3. A method according to claim 2, wherein for said oligopeptide at least 5 amino acids defined by X are valine, leucine or norleucine and any remaining amino acid defined by X is Trp or Tyr.

4. A method according to claim 3, wherein for said oligopeptide at least 5 amino acids defined by X are the same.

5. A method according to claim 1, wherein said oligopeptide is selected from the group consisting of:
(a) Arg-Leu-Leu-Leu-Arg-Leu-Leu-Leu-Gly-Tyr;
(b) Arg-Val-Leu-Leu-Arg-Leu-Leu-Leu-Gly-Tyr;
(c) Arg-Ile-Leu-Leu-Arg-Leu-Leu-Leu-Gly-Tyr;
(d) Arg-Leu-Val-Leu-Arg-Leu-Leu-Leu-Gly-Tyr;
(e) Arg-Leu-Ile-Leu-Arg-Leu-Leu-Leu-Gly-Tyr;
(f) Arg-Leu-Leu-Val-Arg-Leu-Leu-Leu-Gly-Tyr;
(g) Arg-Leu-Leu-Ile-Arg-Leu-Leu-Leu-Gly-Tyr;
(h) Arg-Leu-Leu-Leu-Arg-Val-Leu-Leu-Gly-Tyr;
(i) Arg-Leu-Leu-Leu-Arg-Ile-Leu-Leu-Gly-Tyr;
(j) Arg-Leu-Leu-Leu-Arg-Leu-Val-Leu-Gly-Tyr;
(k) Arg-Leu-Leu-Leu-Arg-Leu-Ile-Leu-Gly-Tyr;
(l) Arg-Leu-Leu-Leu-Arg-Leu-Leu-Val-Gly-Tyr;
(m) Arg-Leu-Leu-Leu-Arg-Leu-Leu-Ile-Gly-Tyr;
(n) Arg-Trp-Leu-Leu-Arg-Leu-Leu-Leu-Gly-Tyr;
(o) Arg-Leu-Trp-Leu-Arg-Leu-Leu-Leu-Gly-Tyr;
(p) Arg-Leu-Leu-Trp-Arg-Leu-Leu-Leu-Gly-Tyr;
(q) Arg-Leu-Leu-Leu-Arg-Trp-Leu-Leu-Gly-Tyr;
(r) Arg-Leu-Leu-Leu-Arg-Leu-Trp-Leu-Gly-Tyr;
(s) Arg-Leu-Leu-Leu-Arg-Leu-Leu-Trp-Gly-Tyr;
(t) Arg-Tyr-Leu-Leu-Arg-Leu-Leu-Leu-Gly-Tyr;
(u) Arg-Leu-Tyr-Leu-Arg-Leu-Leu-Leu-Gly-Tyr;
(v) Arg-Leu-Leu-Tyr-Arg-Leu-Leu-Leu-Gly-Tyr;
(w) Arg-Leu-Leu-Leu-Arg-Tyr-Leu-Leu-Gly-Tyr;
(x) Arg-Leu-Leu-Leu-Arg-Leu-Tyr-Leu-Gly-Tyr;
(y) Arg-Leu-Leu-Leu-Arg-Leu-Leu-Tyr-Gly-Tyr;
and
(z) Arg-nL-nL-nL-Arg-nL-nL-nL-Gly-Tyr.

6. A method according to claim 1, wherein said method further comprises administering at least one additional therapeutic agent selected from the group consisting of anti-diarrheal agents, anti-inflammatory agents and analgesics.

7. A method according to claim 6, wherein said at least one additional therapeutic agent is an anti-diarrheal agent.

8. A method according to claim 6, wherein said immumodulatory peptide and said at least one additional therapeutic agent are administered simultaneously or sequentially.

9. A pharmaceutical preparation for reducing gastrointestinal toxicity induced by cytoablative therapy, comprising:
a) an oligopeptide comprising the sequence
Arg-nL-nL-nL-Arg-nL-nL-nL-Gly-Tyr
wherein nL is norleucine and all amino acids are the D-stereoisomers; and
b) at least one additional therapeutic agent selected from the group consisting of anti-diarrheal agents, anti-inflammatory agents and analgesics.

10. A pharmaceutical preparation according to claim 9, wherein said at least one additional therapeutic agent is an anti-diarrheal agent.

11. A method for improving cytoablative cancer therapy, comprising the administration of an increased maximum tolerated dose of a cytoablative agent in combination with a pharmaceutically effective amount of an immunomodulatory peptide, wherein said immunomodulatory peptide comprises an oligopetide comprising the amino acid sequence
B-X-X-X-B-X-X-X-J-Tyr
wherein
B is Lys or Arg;
X is a aliphatic or aromatic amino acid;
J is Gly, Lys or Arg,
wherein said amino acids are the naturally occurring L-isomers, their D-isomers, and norleucine.

12. A method according to claim 11, wherein for said oligopeptide
B is Arg;
X is an aliphatic non-polar amino acid of from 5 to 6 carbon atoms including norleucine, aromatic amino acid or the D-isomer thereof, there being at least 3 of the same aliphatic non-polar amino acid in said oligopeptide; and
J is Gly or Arg

13. A method according to claim 10, wherein said maximum tolerated dose of said cytoablative agent is increased by at least 1.5× in comparison with the maximum tolerated dose of said cytoablative agent in the absence of said immunomodulatory peptide.

14. A method according to claim 10, wherein said maximum tolerated dose of said cytoablative agent is increased by at least 2× in comparison with the maximum tolerated dose of said cytoablative agent in the absence of said immunomodulatory peptide.

15. A method according to claim 10, wherein for said oligopeptide at least 5 amino acids defined by X are valine, leucine or norleucine and any remaining amino acid defined by X is Trp or Tyr.

16. A method according to claim 10, wherein for said oligopeptide at least 5 amino acids defined by X are the same.

17. A method according to claim 10, wherein said immumodulatory peptide and said cytoablative agent are administered simultaneously or sequentially.

18. A method according to claim 17, wherein said immunomodulatory peptide is administered prior to the administration of said cytoablative agent.
